(19) United States
(12) Patent Application Publication
(10) Pub. No.: US 2021/0222226 A1
(43) Pub. Date: Jul. 22, 2021

(54) METHODS OF MEASURING ENZYME ACTIVITY IN COATING COMPOSTIONS

(71) Applicant: BASF SE, Ludwigshafen an Rhein (DE)

(72) Inventors: Yun Han, San Diego, CA (US); Tuan Tran, San Diego, CA (US)

(21) Appl. No.: 17/251,745

(22) PCT Filed: Jun. 24, 2019
(86) PCT No.: PCT/US2019/038683

§ 371 Date: Dec. 11, 2020

Related U.S. Application Data
(60) Provisional application No. 62/691,404, filed on Jun. 28, 2018.

(51) Int. Cl. C12Q 1/34

(52) U.S. Cl. CPC C12Q 1/34; G01N 2333/942; G01N 2333/924

(57) ABSTRACT

Disclosed herein include methods of detecting and measuring enzymatic activity in coating compositions comprising one or more enzymes contained therein, for example following film formation of the coating compositions. Media-based assays and spectrophotometric-based biochemical assays for analysis of in-film enzymatic activity are provided. Methods of configuring coating compositions to enable spectrophotometric-based analysis are also provided.

### CROSS-REFERENCE TO RELATED APPLICATIONS

The present application claims the benefit of priority to U.S. Application No. 62/691,404, filed Jun. 28, 2018, the contents of which are incorporated in their entirety.

### BACKGROUND

### Field

The present application relates to methods of detecting and measuring enzymatic activity in functionalized coating compositions wherein the biological activity of one or more enzymes contained therein confers one or more desirable properties to a surface (e.g., stain resistance). One aspect relates to media-based methods of detecting in-film enzyme activity. Another aspect relates to spectrophotometric-based biochemical assays of in-film enzyme activity.

### Description of Related Art

Various strategies exist for formulating and testing coating compositions, such as paints, for particular surfaces and applications. However, to date, there has been limited success in assaying functionalized coating compositions for enzymatic activity, particularly the activity of enzymes after film formation occurs (e.g., in-film activity). There remains a need for such assay methods that are inexpensive, easy to perform, are capable of being automated, and work across a broad range of enzymes and coating composition formulations.

### SUMMARY

In several embodiments, methods of measuring an enzyme activity in a coating composition are provided. In some embodiments, the coating composition comprises a paint, a lacquer, a printing ink, a varnish, a shellac, a stain, a textile finish, a sealing compound, a water repellent coating, or any combination thereof. In some embodiments the enzyme is selected from the group comprising an amylase, a lipase, a protease, a laccase, a urease, a mannanase, a cellulase, a xylanase, a formaldehyde dismutase, a phytase, an aminopeptidase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, isomerase, a mannosidase, an oxidase, a pectinase, a peptidoglutaminase, a peroxidase, a polyphenoloxidase, a nuclease, a ribonuclease, a transglutaminase, a xylanase, a pullulanase, an isoamylase, a carrageenase, or any combination thereof.

In some embodiments, the method comprises (a) contacting the coating composition with a surface of a medium, wherein the coating composition comprises an enzyme and the medium comprises a substrate of the enzyme; (b) incubating the medium that is contacted with the coating composition of (a) under a condition to allow the enzyme to react with the substrate in the coating composition; and (c) monitoring one or more physical properties of the medium which is in contact with the coating composition. In some embodiments, a change in at least one of the one or more physical properties (e.g., a color property and/or an optical property) of the medium indicates an activity of the enzyme. In some embodiments, the optical property comprises the opacity and/or transparency of the medium. In some embodiments, one or more of steps (a), (b), or (c) are assisted by automation. In some embodiments, the condition to allow the enzyme to react with the substrate comprises a period of time sufficient to allow the enzyme to react with the substrate, a suitable pH for enzyme activity, a suitable temperature, a suitable moisture level for enzyme activity, or a combination thereof. In some embodiments, the substrate is a chromogenic substrate, a fluorescent substrate and/or a luminescent substrate. In some embodiments, the substrate is a natural substrate. In some embodiments, the substrate is a synthetic substrate. In some embodiments, the substrate comprises milk, casein, azo-barley glucan, azo-carob galactomannan, p-nitrophenyl-B-D-lactopyranoside, red starch, syringaldazine, vegetable oil, azo-xylan, azo-arabinoxylan or any combination thereof. In some embodiments, a product of the enzymatic reaction is a chromogenic product, a fluorescent product, and/or a luminescent product. In some embodiments, the coating composition comprises a film. In some embodiments, the change in the one or more physical properties occurs in the medium underneath the film and/or in the medium surrounding the film. In some embodiments, the film is not contacted with a liquid prior to step (a).

In some embodiments, the medium is substantially flat. In some embodiments, the medium is selected from the group comprising agar, gelatine, poiyvinylalcohol, polyetherglycols, polyethylene glycol monostearate, diethylene glycol distearate, ester wax, polyester wax, nitrocellulose, paraffin wax, and any combination thereof. In some embodiments, the medium further comprises an indicator dye. In some such embodiments, the indicator dye has one or more of the following properties: enhances contrast to facilitate monitoring the opacity of the medium, binds the substrate, binds a product of the enzymatic reaction, and/or is responsive to a change in the pH of the medium resulting from the activity of the enzyme. In some embodiments, the indicator dye is selected from the group comprising thionin, astrazon orange, astrazon blue, toluidine blue, methylene blue, acridine orange, pyronine-G, proflavine, azure A, phloxine B, cresyl violet, safranine O, neutral red, thioflavin T, fast red AL, methylene green, rhodamine B, rhodamine 6G, azure B, indoine blue, brilliant cresyl blue, 4′,6-diamidino-2-phenylindole dihydrochloride hydrate, acridine yellow, acriflavine, pyronin-Y, pyronin-B, meldola's blue, nile blue, nile red, new methylene blue, methyl violet, a triphenylmethane dye, methyl green, crystal violet, victoria blue, brilliant green, basic fuchsin, new fuchsin, ethyl violet, malachite green oxalate, quinaldine red, pinacryptol yellow, pinacyanol bromide, pinacyanol chloride, 2-[4-(dimethylamino)styrl]-1-methylquinolium iodide, 2-[4-(dimethylamino)styrl]-1-methylpyridinium iodide, stains-all, benzopurpurin, methyl green, chlorphenol Red, Bromocresol Green, Bromocresol Purple, Bromothymol Blue, Phenol Red, Thymol Blue, Cresol Red, Alizarin, Mordant Orange, Methyl Orange, Methyl Red, Reichardt's Dye, Congo Red, Eosin Blue, Fat Brown B, Orange G, Metanil Yellow, Naphthol Green B, Methylene Violet 3RAX, Sudan Orange G, Morin Hydrate, Disperse Orange 25, Rosolic Acid, Fat Brown RR, Cyanidin chloride, 3,6-Acridineamine, 6′-Butoxy-2,6-diamino-3,3′-azodipyridine, para-Rosaniline Base, Acridine Orange Base, Carbinol Base, and any combination thereof.

In some embodiments, the method comprises (a) configuring the coating composition to allow a spectrophotometer detection light to pass through the coating composition; (b) placing the coating composition in a sample well of a spectrophotometer, wherein the coating composition comprises an enzyme and the sample well comprises a reaction buffer and a substrate of the enzyme; and (c) monitoring the absorbance at a wavelength under a condition to allow the enzyme to react with the substrate. In some embodiments, a change in the absorbance at the wavelength indicates an activity of the enzyme. In some embodiments, the coating composition comprises a film. In some such embodiments, the film weighs about 1 mg to about 200 mg (e.g., 1, 2, 3, 4, 5, 6, 7, 8, 9, 10, 15, 25, 50, 100, 150, 200, and ranges in between). In some embodiments, the condition to allow the enzyme to react with the substrate comprises one or more of a period of time sufficient to allow the enzyme to react with the substrate, a suitable pH for enzyme activity, and/or a suitable temperature for enzyme activity. In some embodiments, step (a) comprises removing an interior region from the film, wherein the interior region is substantially circular, or other geometrical shape that allows that light pass through the central region of the film. In some embodiments, the film is substantially circular. In some such embodiments, the film has a diameter of about 0.2 cm to about 3.0 cm (e.g., 0.2 cm, 0.4 cm, 0.6 cm, 0.8 cm, 1.0 cm, 1.2 cm, 1.5 cm, 2.0 cm, 2.5 cm, 3.0 cm, and ranges in between). In some embodiments, the interior region has a diameter of about 0.1 cm to about 2.5 cm (e.g., 0.1 cm, 0.2 cm, 0.4 cm, 0.6 cm, 0.8 cm, 1.0 cm, 1.2 cm, 1.5 cm, 2.0 cm, 2.5 cm, and ranges in between).

In some embodiments, step (c) is performed at a temperature of about 4° C. to about 80° C. (e.g., 4° C., 6° C., 8° C., 10° C., 15° C., 20° C., 25° C., 30° C., 35° C., 40° C., 45° C., 50° C., 55° C., 60° C., 65° C., 70° C., 75° C., 80° C., and ranges in between). In some embodiments, step (c) is performed at one or more intervals for a time period of about 2 minutes to about 48 hours (e.g., 2 min, 10 min, 20 min, 30 min, 40 min, 50 min, 1 hr, 2 hr, 4 hr, 6 hr, 8 hr, 12 hr, 16 hr, 20 hr, 24 hr, and ranges in between). In some embodiments, one or more of steps (a), (b), or (c) are assisted by automation.

In some embodiments, the substrate is a natural substrate or a synthetic substrate, wherein the substrate is selected from the group comprising formaldehyde, syringaldazine, 2,2′-Azino-bis(3-Ethylbenzthiazoline-6-Sulfonic Acid), urea, 2-chloro-4-nitrophenyl-maltotrioside, Ala-Ala-Pro-Phe-p-nitrophenyl, p-nitrophenyl-Octanoate, 4-Nitrophenyl-β-D-cellobioside, formaldehyde, azo-carob galactomannan, p-nitrophenyl-B-D-lactopyranoside, azo-carob galactomannan, or p-nitrophenyl-B-D-lactopyranoside or any combination thereof. In some embodiments, the sample well is contained within a multi-well plate comprising a plurality of sample wells. In some such embodiments, the multi-well plate is selected from the group comprising a 6-well microplate, a 12-well microplate, a 24-well microplate, 96-well microplate, and 384-well microplate.

### BRIEF DESCRIPTION OF THE FIGURES

FIG. 1 depicts a schematic representation of a procedure for “harsh” enzyme extraction from dry film according to several embodiments disclosed herein.

FIGS. 2A and 2B depict data related to enzyme activity recovery from Group 1 film and liquid paint samples 1-8 with high enzyme addition (˜0.19% cellulase; ˜48 U/g; Set 3) and low enzyme addition (˜0.0036% cellulase; ˜0.9 U/g; Set 1), respectively. FIG. 2C depicts data related to the protein quantity of extracted Set 3 film samples 1-8 by SDS-PAGE. FIG. 2D depicts data related to the specific activity of extracted Set 3 film samples 1-8. Specific activity=enzyme activity/enzyme quantitation. Paint samples marked “A” and “B” have a pigment volume concentration (PVC) of 40% and 20%, respectively.

FIGS. 3A and 3B depict data related to recovered enzyme activity from “harsh” extraction of Group 2 wet paint samples and dry film samples, respectively. The latex type in the paint formulation is indicated below the sample name in FIG. 3A. The pigment volume concertation (PVC) of the formulations is indicated in FIG. 3B. The relative levels of coalescing agents DPnB and Texanol in the v3 formulations are indicated. Different neutralization agents (NH₃ or NaOH) were used in v6 and v7, as indicated in FIG. 3B. No enzyme was added in v1, v2, v3, v4* and v5* samples.

FIGS. 4A and 4B depict data related to enzyme quantification following “harsh” extraction of Group 2 wet paint samples and dry film samples, respectively. The latex type in the paint formulation is indicated below the sample name in FIG. 4A. The pigment volume concertation (PVC) of the formulations is indicated in FIG. 4B. The relative levels of coalescing agents DPnB and Texanol in the v3 formulations are indicated. The neutralization agents (NH3 or NaOH) used in v6 and v7 are indicated in FIG. 4B.

FIGS. 5A and 5B depict data related to the specific activity of extracted enzyme (cellulase) from Group 2 wet paint samples and dry film samples, respectively.

FIG. 6A depicts a schematic representation of a procedure for measuring in-film enzymatic activity according to several embodiments disclosed herein.

FIG. 6B depicts data related to the in-film enzymatic activity of Group 1 Set 1 paint samples that were assayed according to the procedure outlined in FIG. 6A. Wells with substrate only and film samples loaded with no enzyme were employed as controls.

FIG. 7A depicts a schematic representation of a procedure for “soft” enzyme extraction from film and subsequent activity analysis according to several embodiments disclosed herein. FIG. 7B depicts the cellulase extraction over time of a Group 1 Set 3 3B film sample assayed according to the procedure outlined in FIG. 7A.

FIG. 8A depicts a schematic representation of a procedure for multiple cycles of buffer wash/“soft” enzyme extraction from film and subsequent activity analysis according to several embodiments disclosed herein. FIG. 8B depicts data related to cumulative cellulase activity over 30 minutes (six wash cycles) from a film sample assayed according to the procedure outlined in FIG. 8A. FIG. 8C depicts data related to cellulase activity extraction following six 5-minute washes at room temperature (Washes 1-6) and two 30-minute incubations at 60° C. (Heats 1-2).

FIGS. 9A and 9B depict schematic representations of an in-film total activity assay and a soft extraction assay/in-film residual activity assay, respectively, according to several embodiments disclosed herein. FIG. 9C depicts a 5% agar media containing 0.1% azo-barley glucan incubated with paint film samples that have been loaded with 0.1% cellulase (2, 4, 5, 7, 8, 9, 14, 15, 16, 17) or no enzyme (1, 3, 6, 10, 11, 12, 13) as indicated in Table 5.

FIGS. 10A, 10B, and 10C depict data related to in-film total activity, soft extraction activity, and residual activity of cellulase detected in Group 2 dry film samples, respectively. Latex type of the paint formulation is indicated below the sample name in FIG. 10A. The pigment volume concertation (PVC) is indicated in FIG. 10A. The relative levels of coalescing agents DPnB and Texanol in the v3 formulations are indicated in FIGS. 10A and 10B. The neutralizing agents NH₃ or NaOH present in the v6 and v7 paint formulations, respectively, are indicated in FIG. 10B.

FIG. 11 depicts data related to the mass balance of in-film activity of Group 2 dry film samples. The pigment volume concertation (PVC), latex type, relative levels of coalescing agents, and the neutralizing agents present in the paint formulations are indicated.

FIGS. 12A and 12B depict data related to enzyme activity detected by “harsh” extraction and in-film assays of Group 2 dry film samples, respectively.

FIGS. 13A, 13B, and 13C depicts data related to the in-film enzyme activity of Group 2 dry film samples visualized by the agar plate method after 3, 7, and 22 hours incubation at 37° C., respectively. Agar media containing 5% agar and 0.1% azo-barley glucan were incubated with paint film samples that have been loaded with 0.1% cellulase (2, 4, 5, 7, 8, 9, 14, 15, 16, and 17) or no enzyme (1, 3, 6, 10, 11, 12, and 13).

FIGS. 14A and 14B depict data related to enzyme activity detected by “harsh” extraction and total in-film activity assays of Group 1 Set 1 dry film samples, respectively.

FIGS. 15A and 15B depict data related to enzyme activity detected by total in-film total activity assays and “soft” extraction of Group 1 Set 1A dry film samples, respectively.

FIGS. 16A and 16B depict data related to enzyme activity detected by “harsh” extraction and total in-film activity assays of Group 1 Set 3 dry film samples, respectively.

FIGS. 17A and 17B depict data related to enzyme activity detected by total in-film total activity assays and “soft” extraction of Group 1 Set 3 dry film samples, respectively.

FIG. 18A depicts a schematic representation of the enzyme-catalyzed reaction underlying the red starch agar plate assay according to several embodiments disclosed herein. FIG. 18B depicts a red starch agar plate incubated with the indicated samples at 30° C. for 7 hours.

FIG. 19A depicts a schematic representation of the enzyme-catalyzed reaction underlying the milk agar plate assay according to several embodiments disclosed herein. FIG. 19B depicts a milk agar plate incubated with the indicated samples at 30° C. for 3 hours.

FIG. 20A depicts a schematic representation of the enzyme-catalyzed reaction underlying the vegetable oil agar plate assay according to several embodiments disclosed herein. FIG. 20B depicts a vegetable oil agar plate incubated with the indicated samples at 30° C. for 3 hours. FIG. 20C depicts a vegetable oil agar plate incubated with the indicated samples at 30° C. for 4 hours, with the film removed at the end of the incubation.

FIG. 21A depicts a schematic representation of the enzyme-catalyzed reaction underlying the Syringaldazine (SGZ) agar plate assay according to several embodiments disclosed herein. FIG. 21B depicts a SGZ agar plate incubated with the indicated samples at 30° C. for 4 hours.

FIG. 22A depicts a SGZ agar plate incubated with the indicated films loaded with either 40 U/mL laccase or without enzyme (−Enz). FIG. 22B depicts a milk agar plate incubated with the indicated films loaded with either 0.1% protease or without enzyme (−Enz). FIG. 22C depicts a red starch agar plate incubated with the indicated films loaded with either 1% alpha-amylase or without enzyme (−Enz). FIG. 22D depicts a vegetable oil agar plate incubated with the indicated films loaded with lipase (0.5%, 1%, or 4%) or without enzyme (−Enz). Films comprise a PVC of either 40% (0A samples) or 20% (0B samples). Films without enzyme (−Enz) were added as a negative control. The top and bottom rows depict color and greyscale images of the plate, respectively.

FIG. 23A depicts a schematic representation of the enzyme-catalyzed reaction underlying the colorimetric in-film amylase activity assay according to several embodiments disclosed herein. FIG. 23B depicts the removal of an interior region (of a diameter of 0.31 cm) from a paint film sample (with a 0.6 cm diameter) to allow light to pass through. FIG. 23C depicts the placement of a paint film sample that has been configured to allow light to pass through in a 96-well plate

FIGS. 24A-C depict schematic representations of an in-film total assay, a soft extraction assay and an in-film residual assay (FIGS. 24A, 24B, and 24C, respectively) according to several embodiments disclosed herein.

FIGS. 25A-B depicts SGZ agar plates and milk agar plates (FIGS. 25A and 25B, respectively) incubated with films loaded with either enzyme (“+”; 82.4 U/mL amylase and 0.1% protease, respectively) or without enzyme (“−”). Color and grey scale images are depicted. Films comprise a PVC of either 40% (A & C samples) or 20% (B & D samples) and a filler of Minex 4 (A & B samples) or Celatom (C & D samples).

FIG. 26 depicts data related to the in-film activity of laccase (82.4 U/g) in the indicated film samples. In-film enzyme activity was derived from an in-film total assay, a soft extraction assay or an in-film residual assay as indicated. Films comprise a PVC of either 40% (A & C samples) or 20% (B & D samples) and a filler of Minex 4 (A & B samples) or Celatom (C & D samples).

FIG. 27 depicts data related to the in-film activity of protease (0.2 mg/g) in the indicated film samples. In-film enzyme activity was derived from an in-film total assay, a soft extraction assay or an in-film residual assay as indicated. Films comprise a PVC of either 40% (A & C samples) or 20% (B & D samples) and a filler of Minex 4 (A & B samples) or Celatom (C & D samples).

FIG. 28 depicts data related to the in-film activity of amylase (20 mg/g) in the indicated film samples. In-film enzyme activity was derived from an in-film total assay, a soft extraction assay or an in-film residual assay as indicated. Films comprise a PVC of either 40% (A & C samples) or 20% (B & D samples) and a filler of Minex 4 (A & B samples) or Celatom (C & D samples).

FIG. 29 depicts data related to the in-film activity of lipase (2 mg/g) in the indicated film samples. In-film enzyme activity was derived from an in-film total assay, a soft extraction assay or an in-film residual assay as indicated. Films comprise a PVC of either 40% (A & C samples) or 20% (B & D samples) and a filler of Minex 4 (A & B samples) or Celatom (C & D samples).

FIG. 30 depicts data related to the in-film activity of urease (4 U/g) in the indicated film samples. In-film enzyme activity was derived from an in-film total assay, a soft extraction assay or an in-film residual assay as indicated. Films comprise a PVC of either 40% (v5-40) or 20% (v5-20) and a filler of Duramite.

FIGS. 31A-B depict low and high magnification confocal laser scanning microscopy images of a cross section of paint film comprising Minex filler [(NaK)Al₂(AlSi₃)O₁₀(OH)₂]) and fluorescein-labelled cellulase (FIG. 31A and FIG. 31B, respectively).

FIGS. 32A-B depict low and high magnification confocal laser scanning microscopy images of a cross section of paint film comprising Duramite filler (CaCO₃) and fluorescein-labelled cellulase (FIG. 32A and FIG. 32B, respectively).

FIGS. 33A-B depict low and high magnification confocal laser scanning microscopy images of a bottom view of paint film comprising Minex filler [(NaK)Al₂(AlSi₃)O₁₀(OH)₂]) and fluorescein-labelled cellulase (FIG. 33A and FIG. 33B, respectively).

FIGS. 34A-B depict low and high magnification confocal laser scanning microscopy images of a bottom view of paint film comprising Duramite filler (CaCO₃) and fluorescein-labelled cellulase (FIG. 34A and FIG. 34B, respectively).

FIGS. 35A-B depict confocal laser scanning microscopy visualization of enzyme activity in paint film comprising Minex filler [(NaK)Al₂(AlSi₃)O₁₀(OH)₂]), fluorescein-labelled cellulase and 40% PVC (FIG. 35A) or 20% PVC (FIG. 35B).

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

### Functionalized Coating Compositions

There are provided, in some embodiments, coating compositions and methods for the use of enzymes as components of coating compositions. More specifically, there are provided compositions and methods for incorporating enzymes into coating compositions in a manner to retain one or more enzymatic activities conferred by such enzyme within a paint film. In some embodiments, embedded enzymes retain activity after being directly admixed with a coating composition. Further, in some embodiments, the embedded enzymes retain activity after the coating composition is applied to a surface. In some such embodiments, the one or more enzymes retain activity after film formation occurs (e.g., retains in-film enzymatic activity). In some embodiments, the in-film activity of an embedded enzyme renders the surface bioactive. Provided herein, in several embodiments, are methods of detecting and measuring of enzyme activity within the coating compositions disclosed herein after film formation occurs

In some embodiments, the coating composition comprises an architectural coating (e.g., a wood coating, a masonry coating, an artist's coating), an industrial coating (e.g., automotive coating, a can coating, sealant coating, a marine coating), a specification coating (a camouflage coating, a pipeline coating, traffic marker coating, aircraft coating, a nuclear power plant coating), or any combination thereof. In some embodiments, the coating composition comprises a paint. In other embodiments, the coating composition comprises a clear coating. In some embodiments, the clear coating comprises a lacquer, a varnish, a shellac, a stain, a water repellent coating, or any combination thereof. There are provided, in some embodiments, methods of analyzing enzyme activity within any of the types of coating compositions disclosed herein.

In some embodiments, the compositions and methods herein can produce coating compositions with a bioactivity. Provided herein, in several embodiments, are coating compositions wherein an enzyme's activity is conferred to a surface and/or coating composition via the direct incorporation of an enzyme into the coating composition. In some such embodiments, following application to a surface and subsequent film formation, the enzyme maintains a property, alters a property, and/or confers a property to the surface and/or coating composition. In some embodiments, the enzyme retains at least about 2% (e.g., 2%, 3%, 4%, 5%, 6%, 7%, 8%, 9%, 10%, 11%, 12%, 13%, 14%, 15%, 20%, 25%, 30%, 40%, 50%, 75%, 100%, and ranges in between) activity in-film. In some embodiments, there are provided enzymes as components of coating compositions which confer an activity or other advantage to the coating composition related to the enzyme. In some embodiments, about 0.001 wt % to about 70 wt % (e.g. 0.001%, 0.005%, 0.01%, 0.03%, 0.05%, 0.07%, 0.09%, 0.1%, 0.11%, 0.13%, 0.15%, 0.17%, 0.19%, 0.2%, 0.5%, 0.7%, 1%, 2%, 5%, 10%, 20%, 30%, 40%, 50%, 60%, 70%, and ranges in between) of the coating composition comprises one or more enzymes. In some embodiments, the coating composition further comprises a substrate and/or cofactor for the enzyme. In some embodiments, the one or more enzymes comprises an oxidoreductase, a transferase, a hydrolase, a lyase, an isomerase, a ligase, or any combination thereof. In some embodiments, the one or more enzymes comprise a mannanase, a cellulase, an amylase, a lipase, a protease, a laccase, a urease, or any combination thereof. In some embodiments, the application of the coating compositions provided herein to a surface confers one or more of the following properties to the surface and/or coating composition: self-cleaning, stain resistance, stain blocking, tannin blocking, wood adhesion, paint processing aid, formaldehyde abatement, odor abatement, corrosion resistance, anti-microbial, anti-biofilm, de-greasing, de-icing, decontamination, strippable coating, faster curing, and/or lower VOC content. In some embodiments, the one or more enzymes comprises a cellulase and the cellulase enzyme activity confers improved wood adhesion to the coating composition. In some embodiments, the coating composition comprises an oxidase and the oxidase enzyme activity confers tannin blocking, stain resistance, or stain blocking to the coating composition. In some embodiments, the coating composition comprises a laccase, and the laccase enzyme activity confers tannin blocking to the coating composition. In some embodiments, the coating composition comprises a lipolytic enzyme that confers a self-degreasing property to a surface. There are provided, in some embodiments, methods of analyzing enzyme activity within any of the functionalized coating compositions disclosed herein after film formation occurs.

In some embodiments, the coating composition comprises a binder, a pigment, a liquid component, and one or more enzymes. In some embodiments, the coating composition further comprises one or more additives. In several embodiments, the coating composition comprises a combination of various combination groups and individual ingredients. In some embodiments, the formulation comprises, consists essentially of or consists of several or all of the following groups of ingredients: (1) polymers (binders); (2) liquid components; (3) pigments; (4) enzymes; (5) dispersants; (6) coalescing solvents; (7) plasticizers; (8) defoamers; (9) neutralizers; (10) rheology modifiers; (11) wetting agents; (12) dyes; and (13) biocides. In some embodiments, any one of groups (1)-(3) above is provided in a range of about 0.000001% to about 40.0% (e.g., 0.000001%, 0.00001%, 0.0001%, 0.001%, 0.001%, 0.001%, 0.005%, 0.01%, 0.03%, 0.05%, 0.07%, 0.09%, 0.1%, 0.11%, 0.13%, 0.15%, 0.17%, 0.19%, 0.2%, 0.5%, 1%, 3%, 4%, 5%, 10%, 15%, 20%, 30%, 40%, and ranges in between). In some embodiments, any one of groups (4)-(14) above is provided in a range of about 0.000001 to about 20.0% (e.g., 0.000001%, 0.00001%, 0.0001%, 0.001%, 0.001%, 0.001%, 0.005%, 0.01%, 0.03%, 0.05%, 0.07%, 0.09%, 0.1%, 0.11%, 0.13%, 0.15%, 0.17%, 0.19%, 0.2%, 0.5%, 1%, 3%, 4%, 5%, 10%, 15%, 20%, and ranges in between). In some embodiments, only groups (1)-(4) above are provided. In some embodiments, groups (1)-(4) above are provided and the coating composition further comprises a selection of 1, 2, 3, 4, 5, 6, 7, 8, or 9 of groups (5)-(13). In some embodiments, only groups (1), (2), and (4) above are provided. In some embodiments, groups (1), (2), and (4) above are provided, and the coating composition further comprises a selection of 1, 2, 3, 4, 5, 6, 7, 8, or 9 of groups (5)-(13). The percentages provided above for the groups (1)-(13) are provided as % m/m in some embodiments. In other embodiments, these ingredients are provided as % w/w, % m/v, % v/v, % m/w, or % w/v. There are provided, in some embodiments, methods of analyzing enzyme activity within any of the coating composition formulations disclosed herein, including all concentrations of and combinations of ingredient groups (1)-(13).

As disclosed herein, formulating coating compositions of particular ratios and/or amounts of ingredient groups (1)-(13) can result in additive and/or synergistic effects in increasing in-film enzyme activity. In some embodiments, and as illustrated in the Examples, the presence/absence of ingredient groups (1)-(13), the type of ingredient(s) employed for ingredient groups (1)-(13), the concentrations and ratios of ingredient groups (1)-(13), and/or physical and/or functional interactions between ingredient groups (1)-(13) can significantly impact in-film enzyme activity. Given the number permutations possible when formulating functionalized coating compositions comprising a plurality of ingredient groups (1)-(13), there is a significant need for inexpensive, fast and accurate methods of analyzing enzyme activity within coating compositions. It would especially advantageous that such methods be amendable to automation for enabling screening of large numbers of paint formulations to optimize enzyme compatibility. Further, it would be invaluable that such assay methods are compatible with a broad range of enzyme classes and coating composition types.

Several embodiments of the present invention relate to unique methods of assaying the enzyme activity within functionalized coating compositions. The assay methods described herein are especially beneficial for use in the analysis of enzymes embedded in paint formulations disclosed herein, including architectural coatings and industrial coatings. In several embodiments, the assay methods described herein provide one or more of the following advantages: (i) qualitative, semi-quantitative and/or quantitative readouts; (iii) compatibility across broad classes of enzymes (e.g., ureases, mannanases, cellulases, amylase, lipases, protease, and/or laccases); (iv) compatibility across broad ranges of coating composition formulations (e.g. varying PVC levels, varying types and concentrations of fillers, binders and neutralizers); (v) few steps; (vi) low cost; (vii) short incubation periods; (viii) low volumes of starting materials and reagents, enabling simultaneous analysis of multiple samples; (ix) simple mechanical steps amendable to automation; (x) accuracy across a broad range of enzyme concentrations; and (xi) mimicking paint application conditions in a semi-dry state (instead of full immersion of the film in solution). Furthermore, advantageously, in several embodiments, these methods employ dry and semi-dry films not previously processed, eliminating the need to modify the film first or perform an extraction procedure. In some embodiments, these methods enable screening of large libraries of coating composition formulations for selection of the best candidates for further development. In some embodiments, these assays may be employed as quality control methods.

### Media-Based Assays

In some embodiments, media-based assays of enzyme activity within the coating compositions are disclosed herein. In some embodiments, the methods disclosed herein provide for quantitative or semi-quantitative determinations of enzyme activity in a coating composition. In some embodiments, the inventive methods provide for qualitative determinations of enzyme activity. There are provided, in some embodiments, methods of detecting of an enzyme activity in a coating composition, comprising the steps of: (a) contacting the coating composition with a surface of a medium, wherein the coating composition comprises an enzyme and the medium comprises a substrate of the enzyme; (b) incubating the medium contacted with the coating composition of (a) under a condition to allow the enzyme to react with the substrate; and (c) monitoring one or more physical properties of the medium which is in contact with the coating composition. In some embodiments, a change in at least one of the one or more physical properties of the medium indicates an activity of the enzyme. In some embodiments, physical properties comprise a color property and/or an optical property (e.g., opacity and/or transparency of the medium). In some embodiments, the coating composition is a film. In some embodiments, the film is dry. In some embodiments, the film is in a semi-dry state. In some embodiments, the film is not contacted with a liquid prior to step (a). In some embodiments, the change in the one or more physical properties occurs in the medium underneath the film. In some embodiments, the change in the one or more physical properties occurs in the medium surrounding the film. In some embodiments, a zone of clearing (a reduction in the opacity and/or an increase in transparency) around the coating composition (e.g., a film) indicates an activity of the enzyme. In some embodiments, the medium may contain a substrate that makes the media appear “cloudy”, and which upon enzymatic activity on that substrate produces clearing zones. In some embodiments, the enzymatic activity of hydrolytic enzymes (e.g., proteases, amylases) within a coating composition is assayed by including the substrate in an agar plate and scoring for a hydrolytic clear zone. In some such embodiments, indicator dyes are employed to detect the effects of enzyme action (e.g., use of Congo Red to detect the extent of degradation of celluloses and hemicelluloses). In some embodiments, the appearance of a color in the media indicates an activity of an enzyme. In some embodiments, the turn-over of a substrate into a product may generate or remove a chromogenic, fluorescent, luminescent or otherwise detectable compound. In some embodiments, the medium comprises Brilliant Green and/or Rhodamin Red in combination with olive oil (a substrate for lipase) and Congo Red and carboxy methyl cellulose (CMC) (substrate for cellulase). In some embodiments, enzymatic activity is correlated with the change in the one or more physical properties of the medium.

In some embodiments, the condition to allow the enzyme to react with the substrate comprises a period of time sufficient to allow the enzyme to react with the substrate, a suitable pH for enzyme activity, a suitable temperature, a suitable moisture level for enzyme activity, or any combination thereof. In some embodiments, the medium is substantially flat. In some embodiments, the medium comprises or is derived from agar, gelatine, poiyvinylalcohol, polyetherglycols, polyethylene glycol monostearate, diethylene glycol distearate, ester wax, polyester wax, nitrocellulose, paraffin wax, and derivatives and combinations thereof. In some embodiments, the medium is housed in a container. In some embodiments, the container is transparent. In some embodiments, the container is an agar media plate, bioassay tray, or omni-tray. In some embodiments, the size of the container is configured to allow monitoring of a plurality of coating compositions simultaneously.

As used herein, the terms “substrate” or “enzyme substrate” shall be given their ordinary meaning and shall also refer to a substrate for material on which an enzyme acts to produce a reaction product. In some embodiments, the substrate is a chromogenic substrate, a fluorescent substrate and/or a luminescent substrate. As used herein, the term “chromogenic substrate” shall be given its ordinary meaning and shall also refer to a molecule capable of being cleaved or modified by an enzyme which comprises or is coupled to a chromophore. As used herein, the term “chromophore” shall be given its ordinary meaning and shall also refer to a group of atoms within a molecule that is responsible for the absorption properties and/or light emission in the field of the ultraviolet, visible or infrared of this molecule. In some embodiments, these properties result from an ability to absorb the photon energy within a range of the visible spectrum while the remaining wavelengths are transmitted or broadcast. In some embodiments, chromogenic substrate is colored. In some embodiments, chromogenic substrate is colorless. In some embodiments, the chromogenic substrate releases its chromophore under the action of a specific enzyme. In some embodiments, chromogenic substrate needs no additional chemicals present in the medium upon hydrolysis for color production. As used herein, the term “fluorescent substrate” shall be given its ordinary meaning and shall also refer to a molecule capable of being cleaved or modified by an enzyme which comprises or is coupled to a fluorophore. In some embodiments, a fluorescent substrate will produce a fluorescent product upon modification. In some embodiments, the fluorescent substrate releases its fluorophore under the action of a specific enzyme. As used herein, the term “fluorophore” shall be given its ordinary meaning and shall also refer to a group of atoms within a molecule that is responsible for the ability of this molecule to emit light of fluorescence after excitation. As used herein, the term “luminescent substrate” shall be given its ordinary meaning and shall also refer to substrate will produce a luminescence upon enzyme modification. As used herein, the term “luminescence” shall be given its ordinary meaning and shall also refer to any process in which energy is emitted from a material at a different wavelength from that at which it is absorbed. In some embodiments, luminescence may be measured by intensity and/or by lifetime decay. In some embodiments luminescence includes, but is not limited to, fluorescence, phosphorescence, bioluminescence, chemoluminescence, electrochemiluminescence, crystalloluminescence, electroluminescence, cathodoluminescence, mechanoluminescence, triboluminescence, fractoluminescence, piezoluminescence, photoluminescence, radioluminescence, sonoluminescence, and/or thermoluminescence. In some embodiments, the product of the enzymatic reaction is a chromogenic product, a fluorescent product, and/or a luminescent product. In some embodiments, the enzyme causes a substrate to become chromogenic, fluorogenic, and/or lumigenic by directly modifying the chemical structure of the substrate.

In some embodiments, step (b) is performed for a time period of about 20 minutes to about 7 days (e.g., 20 min, 30 min, 40 min, 50 min, 1 hr, 2 hr, 4 hr, 8 hr, 10 hr, 12 hr, 14 hr, 16 hr, 18 hr, 20 hr, 22 hr, 24 hr, 2 days, 3 days, 4 days, 5 days, 6 days, 7 days, and ranges in between). In some embodiments, step (b) is performed at a temperature of about 4° C. to about 60° C. (e.g., 4° C., 7° C., 10° C., 15° C., 20° C., 25° C., 30° C., 35° C., 40° C., 45° C., 50° C., 55° C., 60° C., and ranges in between). In some embodiments, step (b) further comprises removing the coating composition from the medium after the incubation is complete. In some embodiments, step (c) is performed by visual inspection. In some embodiments, step (c) comprises capturing an image of said container with a color, gray scale, fluorescence, and/or luminescence imaging device. In some embodiments, step (c) is performed at one or more intervals. In some embodiments, one or more of steps (a), (b), or (c) are assisted by automation. In some embodiments, the monitoring of the media is performed under ambient light. As used herein, the term “under ambient light” shall be given its ordinary meaning and shall also refer to the visible spectrum, i.e., colors which can be seen and distinguished with the naked eye. However, it is to be understood that the term “under ambient light” includes using a magnification device, if necessary. In some embodiments, the monitoring of the media is performed under UV irradiation. In some embodiments, the monitoring is performed under any irradiation wavelength.

In some embodiments, the substrate is a natural substrate. In some embodiments, the substrate is synthetic substrate. In some embodiments, substrate comprises milk, casein, azo-barley glucan, azo-carob galactomannan, p-nitrophenyl-B-D-lactopyranoside, red starch, syringaldazine, vegetable oil, azo-xylan, azo-arabinoxylan or any combination thereof. In some embodiments, the one or more enzymes assayed within the coating composition include, but are not limited to, an amylase (e.g., an alpha amylase, a beta amylase), a lipase, a protease, a laccase, a urease, a mannanase, a cellulase, a xylanase, a formaldehyde dismutase, a phytase, an aminopeptidase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, isomerase, a mannosidase, an oxidase, a pectinase, a peptidoglutaminase, a peroxidase, a polyphenoloxidase, a nuclease, a ribonuclease, a transglutaminase, a xylanase, a pullulanase, an isoamylase, a carrageenase, or any combination thereof. In some embodiments, enzyme assayed within the coating composition is selected from the group comprising a mannanase, an amylase, a lipase, a protease, a laccase, a xylanase, and any combination thereof. In some embodiments, the enzyme is an amylase and the substrate is red starch. In some embodiments, the enzyme is a protease and the substrate is one or more of milk, casein or hemoglobin. In some embodiments, the enzyme is a mannanase or a cellulase, and the substrate is azo-barley glucan, azo-carob galactomannan, and/or p-nitrophenyl-B-D-lactopyranoside. In some embodiments, the enzyme is a lipase, the substrate is vegetable oil, and the indicator dye is Nile Red. In some embodiments, the enzyme is a laccase and the substrate is syringaldazine. In some embodiments, the enzyme is a xylanase and the substrate azo-xylan, or azo-arabinoxylan

In some embodiments, the enzymatic reaction is indirectly detected. For example, in some embodiments, the media may comprise a pH indicator which is sensitive to the variation in pH induced by the consumption of the substrate and revealing the metabolism of the target microorganisms, including, but not limited to, a chromophore (e.g., bromocresol purple, bromothymol blue, neutral red, aniline blue, bromocresol blue) or a fluorophore (e.g., 4-methylumbelliferone, hydroxycoumarin derivatives, fluorescein derivatives or resorufin derivatives). In some embodiments in which nonchromogenic substrates are employed, one or more indicator dyes are added to the media to detect enzyme activity. In some embodiments, enzyme activity is detected indirectly. In some embodiments, the medium further comprises an indicator dye. In some such embodiments, the indicator dye binds the substrate. In some embodiments, the indicator dye binds a product of the enzymatic reaction. In some embodiments, the indicator dye is responsive to a change in the pH of the medium resulting from the activity of the enzyme. In some embodiments, the indicator dye enhances contrast to facilitate monitoring the opacity of the medium. In some embodiments, the indicator dye is selected from the group comprising thionin, astrazon orange, astrazon blue, toluidine blue, methylene blue, acridine orange, pyronine-G, proflavine, azure A, phloxine B, cresyl violet, safranine O, neutral red, thioflavin T, fast red AL, methylene green, rhodamine B, rhodamine 6G, azure B, indoine blue, brilliant cresyl blue, 4′,6-diamidino-2-phenylindole dihydrochloride hydrate, acridine yellow, acriflavine, pyronin-Y, pyronin-B, meldola's blue, nile blue, nile red, new methylene blue, methyl violet, a triphenylmethane dye, methyl green, crystal violet, victoria blue, brilliant green, basic fuchsin, new fuchsin, ethyl violet, malachite green oxalate, quinaldine red, pinacryptol yellow, pinacyanol bromide, pinacyanol chloride, 2-[4-(dimethylamino)styrl]-1-methylquinolium iodide, 2-[4-(dimethylamino)styrl]-1-methylpyridinium iodide, stains-all, benzopurpurin, methyl green, chlorphenol Red, Bromocresol Green, Bromocresol Purple, Bromothymol Blue, Phenol Red, Thymol Blue, Cresol Red, Alizarin, Mordant Orange, Methyl Orange, Methyl Red, Reichardt's Dye, Congo Red, Eosin Blue, Fat Brown B, Orange G, Metanil Yellow, Naphthol Green B, Methylene Violet 3RAX, Sudan Orange G, Morin Hydrate, Disperse Orange 25, Rosolic Acid, Fat Brown RR, Cyanidin chloride, 3,6-Acridineamine, 6′-Butoxy-2,6-diamino-3,3′-azodipyridine, para-Rosaniline Base, Acridine Orange Base, Carbinol Base, or any combination thereof. In some embodiments, fluorescent indicator dyes are used to monitor pH changes, such as, for example, fluorescein and seminaphthorhodafluors and their derivatives for the pH range 6-9 and LysoSensor, Oregon Green and Rhodol and their derivatives for the pH range 3-7. Indicator dyes whose wavelength of maximum absorption changes as a function of pH also include, in some embodiments, Thymol Blue (approximate useful pH range 1.2-2.8 and 8.0-9.6), Methyl Orange (pH 3.2-4.4), Bromocresol Green (pH 3.8-5.4), Methyl Red (pH4.2-6.2), Bromothymol Blue (pH 6.0-7.6) and Phenol Red (pH 6.8-8.2). In some embodiments, Phenolphthalein (pH 8.2-10.0) turns from colorless to pink as the pH becomes more alkaline.

### Plate-Based Anti-Microbial/Anti-Biofilm Assays

In some embodiments, coating compositions comprising one or more enzymes that confer anti-microbial and/or anti-biofilm properties to the coating composition and/or surface are provided. The anti-microbial and/or anti-biofilm properties may act on any microorganism of interest. Provided here, in several embodiments, are plate-based methods of assaying the anti-microbial and/or anti-biofilm properties of such functionalized coating compositions.

In some embodiments, the media is a solid or semi-solid growth medium inoculated (e.g., swabbed) with a standardized suspension of the microorganism of interest (e.g., a microorganism whose growth is inhibited by an enzyme embedded within a coating composition). In some embodiments, a coating composition (e.g., film) is placed on the inoculated solid or semi-solid growth medium and incubated for a suitable period of time to visualize the presence/absence of growth under and/or surrounding the coating composition. In some embodiments, the plate is incubated under conditions configured to allow for growth of the microorganism being assayed (e.g., presence or absence of nutrients, pH, moisture content, oxidation-reduction potential, temperature, atmospheric gas composition). In some embodiments, the solid or semi-solid growth medium comprises one or more of routine media, selective media, differential media, selective-differential media, enriched media, susceptibility media, anaerobic media and fungal media. In some embodiments, routine media comprises one or more of trypticase soy blood agar, trypticase soy agar, tryptic soy, BHI blood agar, BHI agar, Casman blood, HBT bi-layer media, and standard methods agar. In some embodiments, selective media comprises one or more of, columbia CNA blood, azide blood agar, chocolate selective, Brucella blood, blood SxT, Strep selective I & II, PEA, Bile Esculin agar, Clostridium diffiicle agar, skirrow, CCFA, CLED, Pseudomonas cepacia agar, SxT blood agar, TCBS agar, CIN, Moraxella catarrhalis media, and charcoal selective. In some embodiments, differential media comprises one or more of brilliant green, CYE-Legionella, centrimide, DNA-se, hektoen enteric agar, Jordans tartrate, mannitol salt, LIA, TSI, FLO-Pseudomonas F, TECH-Pseudomonas P, Sellers, starch agar, thermonuclease, Tinsdale agar, McCarthy, LSM, sorbitol-McConkey, MUG-McConkey. In some embodiments, selective and differential media comprises one or more of MacConkey, EMB, Baird Parker, BHI blood with antibiotics, BiGGY-mycologic, CIN, Clostridium difficile agar, McBride, Pseudomonas isolation agar, S—S agar, turgitol 7, and XLD agar. In some embodiments, enriched media comprises one or more of chocolate, GC chocolate, BHI chocolate, Borget Gengou, heart infusion agar, McCarthy, Regan-Lowe, Thayer-Martin, transgrow medium, cysteine tellurite blood, cysteine tellurite heart, BHT, heart infusion, Loefflers, and serum tellurite. In some embodiments, anaerobic media comprises one or more of columbia base, PEA, CAN, LKV, BBE, Brucella, BHI blood base, KBE, McClung-Toabe, oxgall, Schaedlers, and Wilkens-Chalgren. In some embodiments, a fungal media comprises one or more of BHI base, BiGGY, birdseed, corn meal, cotton seed, DTM, sabourauds dextrose, Fuji medium, inhibition mold, Littman oxgall, mycologic, mycophil, Nickersons, SABHI, and trichophytin.

As used herein, the term “microorganism” shall be given its ordinary meaning and shall also refer to any prokaryotic or eukaryotic microscopic organism capable of growing and reproducing in culture medium, including but not limited to, one or more of bacteria (e.g., motile or vegetative, Gram positive or Gram negative), bacterial spores or endospores, and fungi (e.g., yeast, filamentous fungi, fungal spores). In some embodiments, the assayed microorganisms are pathogenic. As used herein, the term “pathogen” shall be given its ordinary meaning and shall also refer to any pathogenic microorganism, for example, members of the family Enterobacteriaceae, or members of the family Micrococcaceae, or the genera Staphylococcus spp., Streptococcus spp., Pseudomonas spp., Enterococcus spp., Salmonella spp., Legionella spp., Shigella spp., Yersinia spp., Enterobacter spp., Escherichia spp., Bacillus spp., Listeria spp., Campylobacter spp., Acinetobacter spp., Vibrio spp., Clostridium spp., and Corynebacteria spp. In some embodiments, the pathogens can comprise, but are not limited to, Escherichia coli including enterohemorrhagic E. coli e.g., serotype O157:H7, Pseudomonas aeruginosa, Bacillus cereus, Bacillus anthracis, Branhamella catarrhalis, Salmonella enteritidis, Salmonella typhimurium, Listeria monocytogenes, Clostridium botulinum, Clostridium perfringens, Staphylococcus aureus, methicillin-resistant Staphylococcus aureus, Campylobacter jejuni, Yersinia enterocolitica, Vibrio vulnificus, Clostridium difficile, vancomycin-resistant Enterococcus, Streptococcus pyogenes, Serratia marcescens, and/or Enterobacter sakazakii.

### Biochemical Assays

Biochemical analysis of coating compositions by spectrophotometric methods are provided in some embodiments. In some embodiments, the methods disclosed herein provide for quantitative or semi-quantitative determinations of enzyme activity in a coating composition. In some embodiments, the methods provide for qualitative determinations of enzyme activity. In some embodiments, the biochemical assay is performed with a spectrophotometer. In some embodiments, the biochemical assay is performed with a fluorimeter. In some embodiments, the biochemical assay is performed with a luminometer. There are provided, in some embodiments, methods of measuring an enzyme activity in a coating composition, comprising the steps of: (a) configuring the coating composition to allow a spectrophotometer detection light to pass through the coating composition; (b) placing the coating composition in a sample well of a spectrophotometer, wherein the coating composition comprises an enzyme and the sample well comprises a reaction buffer and a substrate of the enzyme; and (c) monitoring the absorbance at a wavelength under a condition to allow the enzyme to react with the substrate. In some embodiments of the methods provided here, step (a) is omitted. In some embodiments, a change in the absorbance at the wavelength indicates an activity of the enzyme. In some embodiments, the condition to allow the enzyme to react with the substrate comprises a period of time sufficient to allow the enzyme to react with the substrate, a suitable pH for enzyme activity, a suitable temperature for enzyme activity, or a combination thereof. In some embodiments, the wavelength is between about 400 and about 700 nm (e.g., 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, and ranges in between). In some embodiments, step (c) is performed at a temperature of about 4° C. to about 80° C. (e.g., 4° C., 5° C., 6° C., 7° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., 60° C., 62° C., 64° C., 66° C., 68° C., 70° C., 72° C., 74° C., 76° C., 78° C., 80° C., and ranges in between). In some embodiments, step (c) is performed at more one or more intervals for a time period of about 2 minutes to about 48 hours (e.g., 2 min, 4 min, 8 min, 10 min, 12 min, 14 min, 16 min, 18 min, 20 min, 22 min, 24 min, 26 min, 28 min, 30 min, 32 min, 34 min, 36 min, 38 min, 40 min, 45 min, 50 min, 55 min, 1 hr, 2 hr, 4 hr, 6 hr, 8 hr, 10 hr, 15 hr, 20 hr, 25 hr, 30 hr, 35 hr, 40 hr, 45 hr, 48 hr, and ranges in between). In some embodiments, one or more of steps (a), (b), or (c) are assisted by automation.

In some embodiments, the coating composition comprises a film. In some embodiments, the film weighs about 1 mg to about 200 mg (e.g., 1 mg, 2 mg, 3 mg, 4 mg, 5 mg, 6 mg, 7 mg, 8 mg, 9 mg, 10 mg, 12 mg, 14 mg, 16 mg, 18 mg, 20 mg, 40 mg, 60 mg, 80 mg, 100 mg, 120 mg, 140 mg, 160 mg, 180 mg, 200 mg, and ranges in between). In some embodiments, the weight of the film is selected based on the format of the well plate. In some embodiments, the weight of the film is selected based on the thickness of the film. In some embodiments, the film is substantially circular. In some embodiments, the geometric shape of the film is selected based on the thickness of the film. In some embodiments, the geometric shape of the film is selected based on the shape of the sample well. In some embodiments, the film has a diameter of about 0.1 cm to about 3.0 cm (e.g., 0.1 cm, 0.2 cm, 0.3 cm, 0.4 cm, 0.5 cm, 0.6 cm, 0.7 cm, 0.8 cm, 0.9 cm, 1.0 cm, 1.2 cm, 1.4 cm, 1.6 cm, 1.8 cm, 2.0 cm, 2.2 cm, 2.4 cm, 2.6 cm, 2.8 cm, 3.0 cm, and ranges in between).

In some embodiments, the film is dry or in a semi-dry state prior to step (a). In some embodiments, the film is not contacted with a liquid prior to step (a). In some embodiments, the methods provided herein require an incident light path to pass through the sample well and a measurement the absorbance of that light. In some embodiments, measurement of absorbance within sample wells comprising unaltered dry paint films is not feasible, as the dry paint film blocks the light path. In some such embodiments, the light blockage causes problems, including, but not limited to, an inaccurate readout of enzyme activity, an extended assay period required, higher levels of substrate required, higher levels of enzyme required, and/or incompatibility of particular paint formulations with the assay. Provided herein is a solution to this problem: configuring the film to allow the incident light path to pass through, such as, for example, by removing an interior portion of the film before it is placed in the sample well. By way of example, in some embodiments, dry paint films containing enzymes are cut into an “O-ring shape” pieces using two different sizes of hole punchers. In one embodiment, a hole puncher (e.g., with a 0.6 cm diameter) cuts the dry paint film into a circular piece that is configured to fit into a well of a 96-well plate. In still further embodiments, this circular piece of dry paint film is further cut with a smaller hole puncher (e.g., a diameter=0.31 cm) at the center. In some such embodiments, the end result is a 0.6 cm disk with a 0.31 cm hollow center (or “O-ring shape”) that allows the incident light to pass through the center of each well. In some such embodiments, this configuring of the paint film enables recording of the absorbance of that light while also allowing the enzymes in “O-ring” portion of the paint film to interact with the substrate solution added to the well. Importantly, in some embodiments, this method allows detection of enzyme activity from enzyme that was released into the solution from the paint film as well as immobilized enzyme in the dry paint film. In some embodiments, step (a) comprises removing an interior region from the film. In some embodiments, the interior region is substantially circular. In some embodiments, the geometric shape of the interior region is selected based on the thickness of the film. In some embodiments, the geometric shape of the interior region is selected based on the shape of the sample well. In some embodiments, the interior region has any geometrical shape that allows that light pass through. In some embodiments, the interior region has a diameter of about 0.05 cm to about 2.5 cm (e.g., 0.05 cm, 0.075 cm, 0.1 cm, 0.2 cm, 0.3 cm, 0.4 cm, 0.5 cm, 0.6 cm, 0.7 cm, 0.8 cm, 0.9 cm, 1.0 cm, 1.2 cm, 1.4 cm, 1.6 cm, 1.8 cm, 2.0 cm, 2.2 cm, 2.4 cm, 2.5 cm, and ranges in between). In some embodiments, the sample well is contained within a multi-well plate comprising a plurality of sample wells. In some embodiments, the multi-well plate comprises a microplate. Multi-well plates provided herein include, but are not limited to, that that have between about 6 and about 5,000 wells, preferably between about 96 and about 4,000 wells, most preferably in multiples of 96. In some embodiments, the multi-well plate is selected from the group comprising a 6-well microplate, a 12-well microplate, a 24-well microplate, a 96-well microplate, and a 384-well microplate.

In some embodiments, a substrate is selected that produces color change upon enzymatic reaction. In some embodiments, the substrate is a chromogenic substrate, a fluorescent substrate and/or a luminescent substrate. In some embodiments, the product of the enzymatic reaction is a chromogenic product, a fluorescent product, and/or a luminescent product. In some embodiments, the enzyme causes a substrate to become chromogenic, fluorogenic, and/or lumigenic by directly modifying the chemical structure of the substrate. In some embodiments, the substrate is a natural substrate. In some embodiments, the substrate is synthetic substrate. In some embodiments, substrate is selected from the group comprising syringaldazine, 2,2′-Azino-bis(3-Ethylbenzthiazoline-6-Sulfonic Acid), urea, 2-chloro-4-nitrophenyl-maltotrioside, Ala-Ala-Pro-Phe-p-nitrophenyl, p-nitrophenyl-Octanoate, 4-Nitrophenyl-β-D-cellobioside, formaldehyde, azo-carob galactomannan, p-nitrophenyl-B-D-lactopyranoside, azo-carob galactomannan, or p-nitrophenyl-B-D-lactopyranoside or any combination thereof. In some embodiments, the one or more enzymes assayed within the coating composition include, but are not limited to, an amylase (e.g., an alpha amylase, a beta amylase), a lipase, a protease, a laccase, a urease, a mannanase, a cellulase, a xylanase, a formaldehyde dismutase, a phytase, an aminopeptidase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, isomerase, a mannosidase, an oxidase, a pectinase, a peptidoglutaminase, a peroxidase, a polyphenoloxidase, a nuclease, a ribonuclease, a transglutaminase, a xylanase, a pullulanase, an isoamylase, a carrageenase, or any combination thereof. In some embodiments, enzyme assayed within the coating composition is selected from the group comprising a mannanase, an amylase, a formaldehyde dismutase, a lipase, a protease, a laccase, a xylanase, a urease, and any combination thereof.

In some embodiments, the enzyme is a laccase, the substrate is syringaldazine, and the wavelength is between about 400 and about 600 nm (e.g., 400 nm, 420 nm, 440 nm, 460 nm, 480 nm, 500 nm, 520 nm, 540 nm, 560 nm, 580 nm, 600 nm, and ranges in between). In some such embodiments, the reaction buffer comprises about 100 mM K₂PO₄ (pH 6-8).

In some embodiments, the enzyme is an alpha amylase, the substrate is 2-chloro-4-nitrophenyl-maltotrioside, and the wavelength is between about 350 to about 450 nm (e.g., 350 nm, 360 nm, 380 nm, 400 nm, 420 nm, 440 nm, 450 nm, and ranges in between). In some such embodiments, the reaction buffer comprises about 1 unit of β-glucosidase. In some such embodiments, the reaction buffer further comprises about 50 mM HEPES (pH 6-8), about 50 mM sodium phosphate (pH 6-8), or about 50 mM TRIS (pH 6-8).

In some embodiments, the enzyme is a urease, and the substrate is urea. In some such embodiments, the product of the substrate is ammonia. In some such embodiments, the addition of a detecting agent (e.g., Berthelot's reagent (with an alkaline solution of phenol and/or hypochlorite)) reacts with ammonia and generates a blue color. In some such embodiments, the wavelength is between about 600 and about 700 nm (e.g., 600 nm, 620 nm, 640 nm, 660 nm, 680 nm, 700 nm, and ranges in between). In some such embodiments, the reaction buffer comprises about 10 mM sodium phosphate (pH 7).

In some embodiments, the enzyme is a protease, the substrate is Ala-Ala-Pro-Phe-p-nitrophenyl, and the wavelength is between about 350 to about 450 nm (e.g., 350 nm, 360 nm, 380 nm, 400 nm, 420 nm, 440 nm, 450 nm, and ranges in between). In some such embodiments, the reaction buffer comprises about 50 mM HEPES (pH 6-8), about 50 mM sodium phosphate (pH 6-8), or about 50 mM TRIS (pH 6-8).

In some embodiments, the enzyme is a lipase, the substrate is p-nitrophenyl-Octanoate, and the wavelength is between about 350 to about 450 nm (e.g., 350 nm, 360 nm, 380 nm, 400 nm, 420 nm, 440 nm, 450 nm, and ranges in between). In some such embodiments, the reaction buffer comprises about 50 mM HEPES (pH 6-8), about 50 mM sodium phosphate (pH 6-8), or about 50 mM TRIS (pH 6-8). In some such embodiments, the reaction buffer further comprises about 0.001% Triton-X100, about 100 nM NaCl, and about 20 mM CaCl₂).

In some embodiments, the enzyme is a mannanase or cellulase, the substrate is 4-Nitrophenyl-β-D-cellobioside, and the wavelength is between about 350 to about 450 nm (e.g., 350 nm, 360 nm, 380 nm, 400 nm, 420 nm, 440 nm, 450 nm, and ranges in between). In some such embodiments, the reaction buffer comprises about 50 mM HEPES (pH 6-8), about 50 mM sodium phosphate (pH 6-8), or about 50 mM TRIS (pH 6-8).

In some embodiments, the enzyme is a formaldehyde dismutase and the substrate is formaldehyde. In some such embodiments, the product changes the pH of the assay solution and a pH-sensitive fluorescent reagent (e.g., fluorescein) is added to the reaction buffer. As a result, in some such embodiments, the fluorescence of fluorescein changes according to the change in pH wherein the emission wavelength is between about 500 and about 600 nm (e.g., 500 nm, 520 nm, 540 nm, 560 nm, 580 nm, 600 nm, and ranges in between). In some such embodiments, the reaction buffer comprises about 0.35 μg/mL of fluorescein. In some such embodiments, the reaction buffer further comprises about 2 mM HEPES (pH 7-8), about 2 mM sodium phosphate (pH 7-8), or about 2 mM TRIS (pH 7-8).

### EXAMPLES

### Example 1: Optimization of Enzyme Extraction from Liquid Paint and Dry Film

In this example, enzyme extraction methodology and an optimal protocol were developed for maximal recovery of enzyme activity from liquid paint and dry film. The following extraction factors were tested: pH: (7.5, 8.5, and 10.0); detergent (Triton X-100) concentration (at 0%, 0.25%, 0.5%, and 1.0%); salt concentration (0, 100, 500 and mM); BSA concentration (0, 0.1%, and 1.0%); temperature (RT, 40° C., 60° C., and 80° C.); and incubation time: (15, 30, 60, and 120 minutes). Table 1 depicts the paint samples (wet paint and dry film) employed in the extraction optimization studies, which vary with regards to both PVC levels and filler chemistry. Paint samples were loaded with cellulase/mannanase Pyrolase HT® at the indicated concentrations.

TABLE 1
PAINT SAMPLES USED FOR EXTRACTION OPTIMIZATION

| Sample | Enzyme | Activity | | | |
|---|---|---|---|---|---|
| # | Loading (wt %) | (U/g) | PVC | Filler Chemistry | Filler Product |
| 4A-3 | 0.19% | 47.8 | 40 | Mg₃Si₄O₁₀(OH)₂ | Mistron 400C |
| 4B-3 | 0.19% | 47.8 | 20 | Mg₃Si₄O₁₀(OH)₂ | Mistron 400C |
| 8A-3 | 0.19% | 47.8 | 40 | BaSO₄ | Barimite 200 |
| 8B-3 | 0.19% | 47.8 | 20 | BaSO₄ | Barimite 200 |

### Methods

For sample extraction, 50 mg of wet paint or film was weighed out and added to 500 μL of buffer (10× extraction ratio). The sample was agitated by shaking for 1 hour at the designated time and temperature. Liquid control samples were diluted and also treated in the same way. Samples were centrifuged and the supernatant was further analyzed by enzyme activity assay and protein quantification. The enzyme substrate employed was Resorufin Cellobioside (0.1 mM in reaction). All the extracted samples were diluted 50× for the assay with a dilution buffer comprising 50 mM MES buffer, pH 6, and 0.5% Triton X-100. The assay was performed at temperature of 25° C., and the enzyme activity was detected with an excitation wavelength of 550 nm and an emission wavelength of 590 nm. Enzyme quantity was determined by SDS-PAGE. Relative specific activity was calculated by the ratio of enzyme activity:quantity.

### Results and Conclusions of Enzyme Extraction Studies

For enzyme extraction from dry film samples, a high pH and high temperature were found to improve extraction, and higher detergent (Triton X-100) concentration also improved extraction. It was found that the use of NaCl and BSA did not increase enzyme extraction from dry film. While it was discovered that higher temperature extracts enzyme faster from film, it also leads to loss of activity over time. For enzyme extraction from wet paint samples, it was discovered that full enzyme activity was easily extracted and recovered in very short extraction time. pH, temperature, use of detergent (Triton X100), NaCl and BSA had little effect. Additionally, it was found that higher temperatures and longer extraction times resulted in lower recovered specific activity. The studies provide proof of concept that enzymes directly embedded in wet paint retain their activity, and further that they remain active following subsequent extraction from film.

Based on these investigations, the following optimized extraction conditions (for “harsh” extraction) were derived: 1) an extraction solution comprising 50 mM CAPS Buffer, pH 10, 0.5% Triton; 2) an extraction ratio of 10× (500 μl extraction solution added to 50 mg of wet paint or dry film); 3) an incubation time of 30 minutes shaking; and 4) an incubation temperature of 60° C. for dry film and room temperature for wet paint. The extraction mixture is centrifuged at 30,000 g for 5 minutes, and supernatant is then analyzed for enzyme activity and protein quantity.

Procedure for Enzyme Extraction from Dry Film (“Harsh Extraction”) and Enzyme Analysis from the Extract

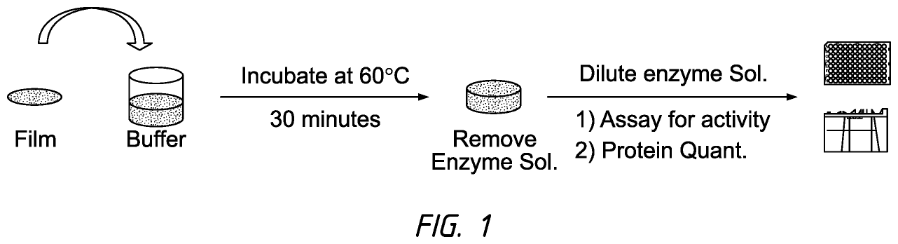

Based on the foregoing investigations the following “harsh” enzyme extraction protocol (with elevated temperature & pH) was developed. Following incubation at 60° C. for 30 minutes in 50 mM CAPS buffer (with 0.5% Triton-X100, pH 10), the enzyme solution is removed, diluted, and assayed for activity and protein quantification. Activity is determined using Resorufin Cellobioside as substrate (in 50 mM MES Buffer with 0.25% Triton-X100, pH 6 at room temperature) while protein quantification is performed by SDS-PAGE. FIG. 1 depicts a schematic representation of a procedure for “harsh” enzyme extraction from dry film according to several embodiments disclosed herein.

### Example 2: Use of Paint Samples with Different Ingredient Matrices to Elucidate the Impact of Paint Formulations on Enzyme Activity Extraction

In this example, different paint formulations (e.g. PVC, fillers, pH, latex chemistry, additive chemistry . . . ) were used to understand the mechanism of enzyme recovery loss and identify the components compatible or not compatible with enzyme in wet paint and dry paint films. Paint samples were loaded with cellulase/mannanase Pyrolase HT® at the indicated concentrations.

### Group 1

Table 2 depicts the Group 1 samples used to understand the impact of paint ingredients on enzyme activity recovery. The “Set 1” and “Set 3” samples were loaded with low and high levels of enzymes, respectively. The listed enzyme loading and activity in Table 2 are targets in wet paint samples; these target levels in corresponding dry film samples are expected to be doubled due to drying.

TABLE 2
PAINT FORMULATION - GROUP 1 PAINT SAMPLES

| | Set 1 | | Set 3 | | | | |
|---|---|---|---|---|---|---|---|
| | Enzyme | | Enzyme | | Paint Formulation | | |
| Sample | loading | Activity | loading | Activity | | | Filler |
| # | (wt %) | (u/g) | (wt %) | (u/g) | PVC | Filler Chemistry | Product |
| OA | 0.0000% | 0.00 | | | 40 | (NaK)Al2(AlSi3)O10(OH)2 | Minex 4 |
| OB | | | | | 20 | | |
| 1A | 0.0036% | 0.90 U/g | 0.19% | 47.8 U/g | 40 | CaCO3 | Durannite |
| 1B | | | | | 20 | | |
| 2A | | | | | 40 | Al2Si2O5(OH)4 | ASP 172 |
| 2B | | | | | 20 | | |
| 3A | | | | | 40 | Al2Si2O5(OH)4 | Mattex |
| 3B | | | | | 20 | | Pro |
| 4A | | | | | 40 | Mg3Si4O10(OH)2 | Mistron |
| 4B | | | | | 20 | | 400c |
| 5A | | | | | 40 | SiO2 | Celatom |
| 5B | | | | | 20 | | |
| 6A | | | | | 40 | KAl2(AlSi3)O10(OH)2 | Mica WG |
| 6B | | | | | 20 | | 325 |
| 7A | | | | | 40 | (NaK)Al2(AlSi3)O10(OH)2 | Minex 4 |
| 7B | | | | | 20 | | |
| 8A | | | | | 40 | BaSO4 | Barimite |
| 8B | | | | | 20 | | 200 |

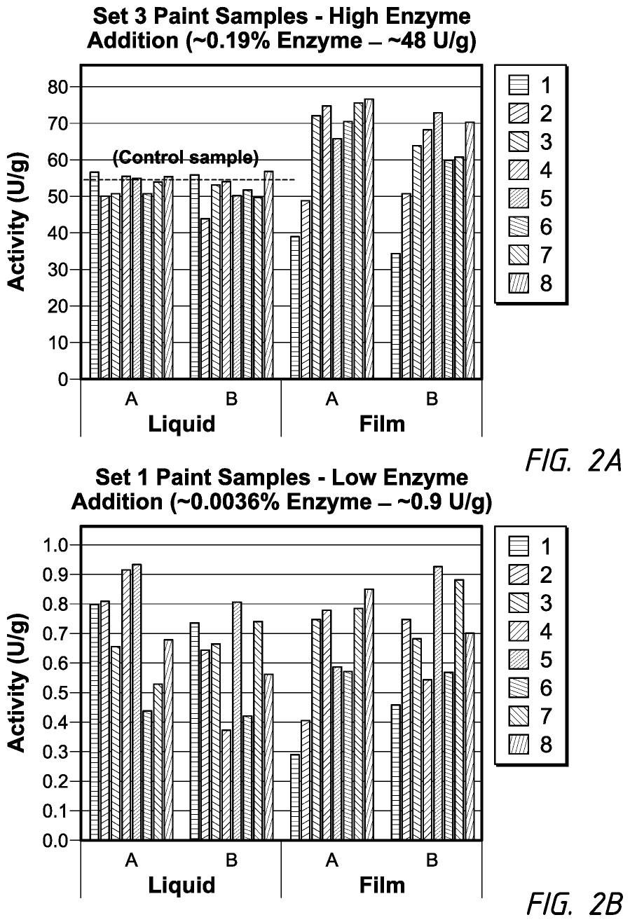

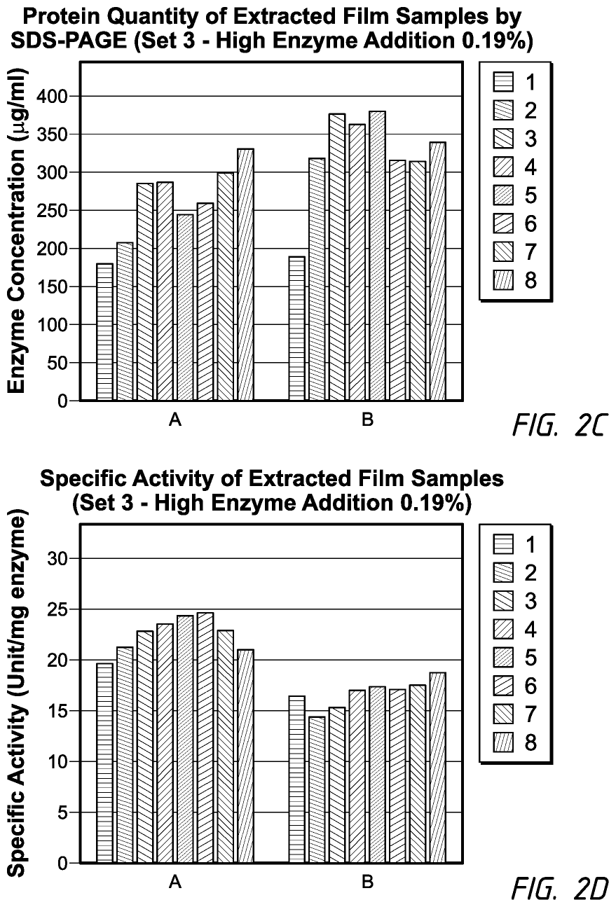

Wet paint samples with high enzyme loading (Set 3) showed near complete enzyme recovery. On the other hand, dry film samples with high enzyme addition showed overall lower recovery than wet paint; however, still more than 50% recovery was observed for most samples in Set 3 (FIG. 2A). Recovery from samples with low enzyme addition (Set 1) showed more variations among samples, with lower recovery found in the dry film samples. (FIG. 2B). PVC level did not appear to have any obvious effect of enzyme activity recovery. Interestingly, filler chemistry impacted enzyme extractability of dry film samples, as CaCO3 (Duramite)—Sample 1 and Al2Si2O5(OH)₄ (ASP172)—Sample 2 exhibited the lowest recovery.

FIG. 2C shows the protein quantity of extracted Set 3 film samples and FIG. 2D shows the specific activity (enzyme activity/enzyme quantitation) of these samples. There was no obvious difference in specific activity observed among the samples, with values found to be very close to the control enzyme sample, indicating the extracted enzyme from different film samples is as active as the control enzyme. The difference between sample sets “A” and “B” (pigment volume concentration (PVC) of 40% and 20%, respectively) is likely due to experimental variation.

### Group 2

A second group of paint formulation—Group 2—is depicted in Table 3. The Group 2 paint samples include 17 different formulations: 10 samples with enzyme loading (0.1% in wet paint and ˜0.2% Enzyme in dry film) and 7 control samples with no enzyme addition. The v1-v3 samples are similar to industrial coating formulations: they have a low PVC level and contain different Joncryl latex that is rigid and requires coalescing agents (such as DPnB and Texanol) for film formation. The v6-v7 Samples are similar to the Group 1 paint samples and comprise CaCO₃ (Duramite) filler, different VOC levels, and different neutralizing agent types (NaOH vs NH₃) and concentrations.

TABLE 3
PAINT FORMULATION - GROUP 2 PAINT SAMPLES

| | Sample | | | | | |
|---|---|---|---|---|---|---|
| | 2 | 4 | 5 | 7 | 8 | 9 |
| Name | v1_E230 | v2_E230 | v2_E200 | v3_E230 | v3_E200 | v3_E100 |
| Latex | Joncryl | Joncryl | Joncryl | Joncryl | Joncryl | Joncryl |
| | 537 | 1522 | 1522 | 1524 | 1524 | 1524 |
| Neutralizer | | | | | | |
| Water | 68.6 | 68.6 | 68.6 | 68.6 | 68.6 | 68.6 |
| Neutralizer | | | | | | |
| Dispex CX 4230 | 2.0 | 1.9 | 1.9 | 1.9 | 1.9 | 1.9 |
| Dispex Ultra FA 4416 | 0.5 | 0.4 | 0.4 | 0.4 | 0.4 | 0.4 |
| FoamStar ST 2446 | 2.4 | 2.4 | 2.4 | 2.4 | 2.4 | 2.4 |
| Ti-Pure R-900 | 140.1 | 140.1 | 140.1 | 140.1 | 140.1 | 140.1 |
| Grind for 20 min | | | | | | |
| Water | 18.97 | 18.97 | 18.97 | 18.97 | 18.97 | 18.97 |
| Dipropylene glycol n- | 60 | 60 | 50 | 60 | 50 | 22 |
| butyl ether (DPnB) | | | | | | |
| Texanol | 30 | 30 | 25 | 30 | 25 | 11 |
| Hydropalat WE 3322 | 2 | 2 | 2 | 2 | 2 | 2 |

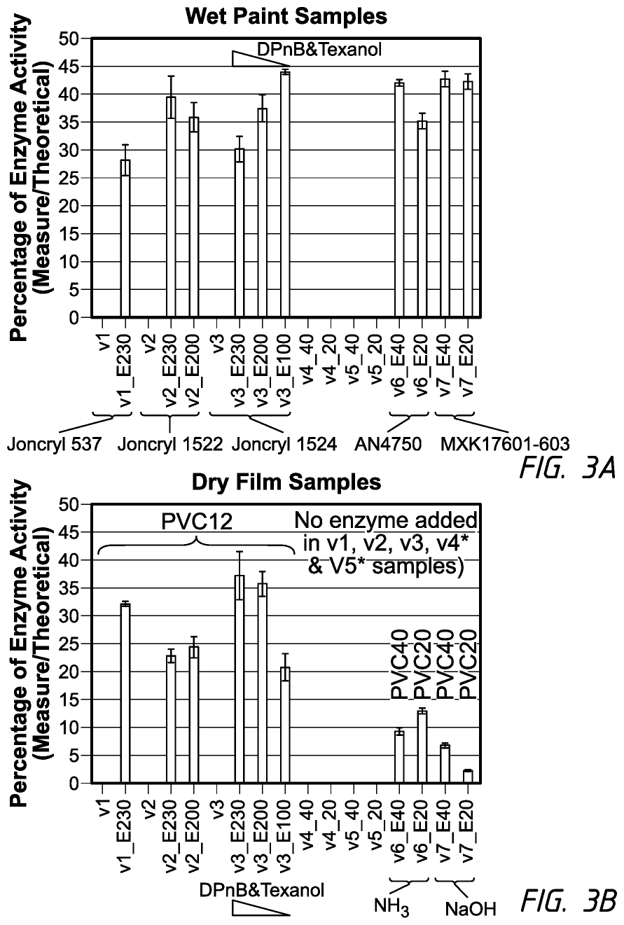

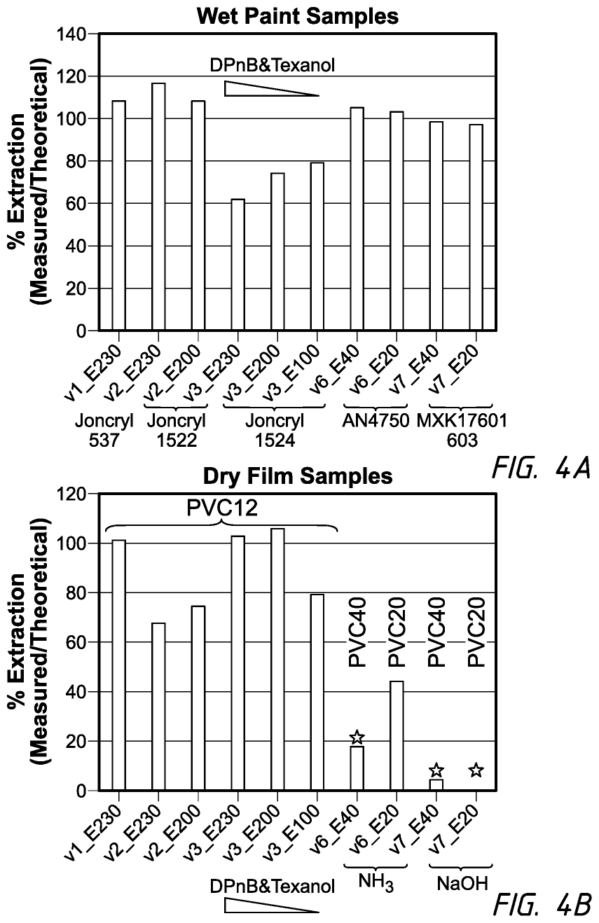

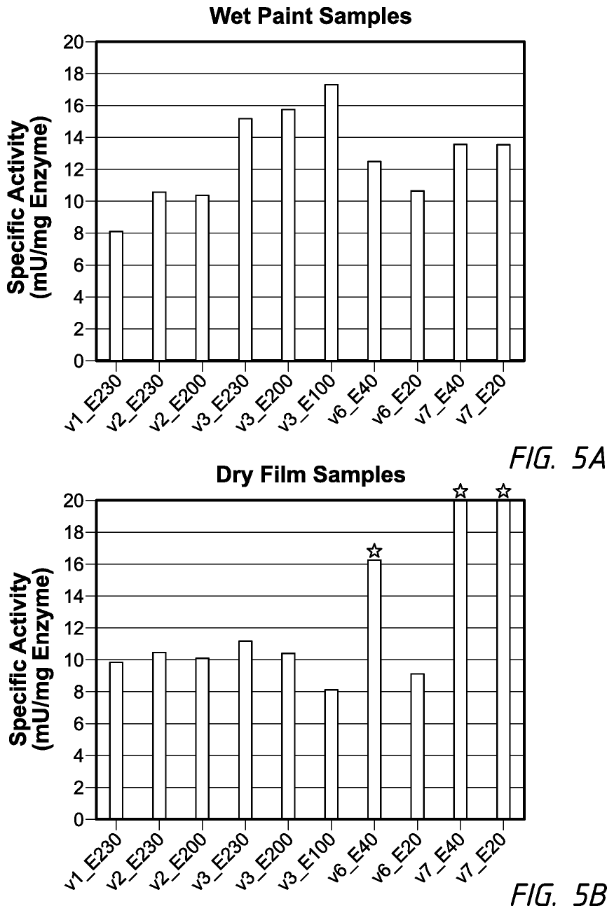

FIGS. 3A and 3B show recovered enzyme activity from “harsh” extraction of Group 2 wet paint samples and dry film samples, respectively. Approximately 30-45% of enzyme activity is recovered by “harsh extraction” for wet paint samples. Unexpectedly, coalescing agents DPnB and Texanol were found to affect activity extraction efficiency, but exhibited opposite trends in wet paint samples versus dry film samples. The v6 and v7 samples exhibited lower extracted activity as dry film compared to v1, v2 and v3 samples; and higher extractability was found from film samples comprising NH₃ than those with NaOH as the neutralizing agent. Enzyme quantification following “harsh” extraction of the wet paint samples and dry film samples is shown in FIGS. 4A and 4B, respectively. Extraction efficiency ranged from 60-100%, with wet paint showing more consistent extraction (100% extracted). Extractability was found to decrease with increasing levels of coalescing agents DPnB and Texanol. For the dry paint samples analyzed, lower amounts of the enzyme were extracted from the v6 and v7 samples. Additionally, higher extractability was observed from film samples comprising NH₃ than those with NaOH. FIGS. 5A and 5B depict data related to the specific activity of extracted enzyme (cellulase) from Group 2 wet paint samples and dry film samples, respectively. The specific activity of wet paint film samples ranged from 8-18 mU/mg. For dry film samples, similar specific activity values (8-11 mU/mg) are observed (except for very high values from v6 and v7 samples, possibly due to inaccurate protein quantification from the very low enzyme recovery samples shown in FIG. 4B). The reference specific activity of the cellulase tested is ˜18 mU/mg.

The studies above indicate that multiple formulations components affect enzyme extractability. Overall lower enzyme extraction and slightly lower specific activity of extracted enzyme was observed among the Group 2 samples as compared to Group 1 samples. Coalescing agents (DPnB and Texanol) were found to affect extraction efficiency, with opposite trends in wet paint samples versus dry film samples. Decreasing activity was observed with increasing coalescing agent content in wet paint, possibly due to mild enzyme inactivation by the organic solvent. The increasing activity with increasing coalescing agent content in dry paint is possibly due to better film formation (as DPnB and Texanol are evaporated after drying). The v6 and v7 samples tested have low extracted activity in dry film, which confirms the Group 1 finding of lower enzyme extraction from Duramite-containing film. Additionally, switching the neutralizing agent from NH₃ to NaOH was also found to decrease enzyme recovery. PVC level was not found to have any effect on enzyme extraction. These experiments also provide proof of concept that enzymes directly embedded in wet paint retain their activity following film formation.

### Example 3: Development of Assay Procedures to Analyze Enzyme Activity in-Film

In this example, assay protocols were developed to reliably and accurately determine enzyme activity directly in-film (rather than under a “harsh” extraction that favors maximal enzyme recovery). As used herein, in some embodiments, in-film activity refers to direct activity when placing a film under a “native” solution, “soft” extraction refers to enzyme that can be extracted in solution under a more native solution condition (as compared to the “harsh” optimal condition), and residual activity refers to activity left in-film after “soft” extraction. Agar plate assays for visualizing enzyme in-film activity were also developed and tested. A cellulase/mannanase was contained in the film samples.

### Development of an In-Film Total Assay, a Soft Extraction Assay, and an In-Film Residual Assay

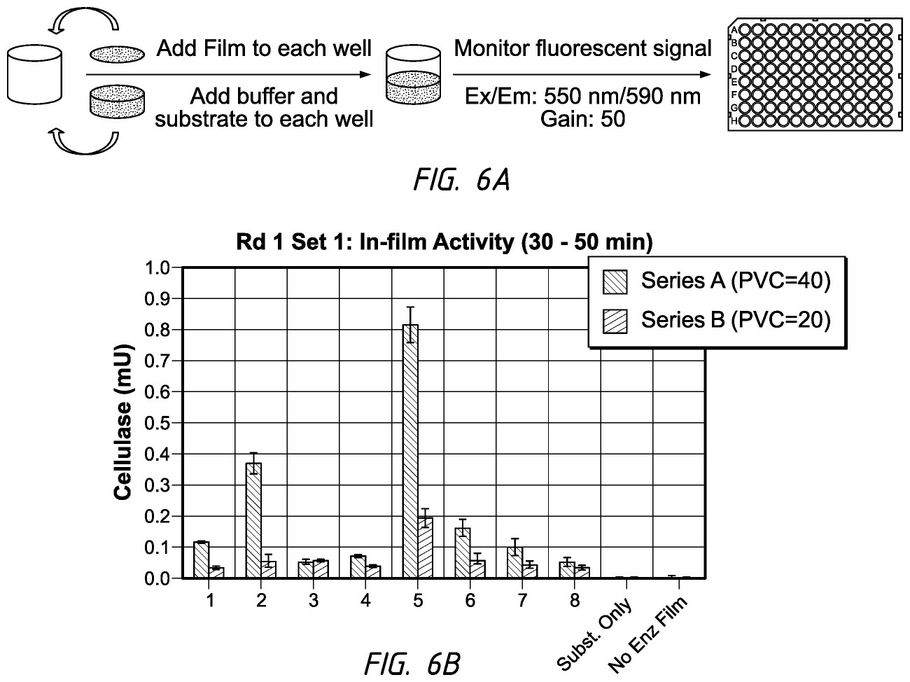

An assay for directly measuring in-film enzymatic activity without initial “harsh extraction” was developed (FIG. 6A). Approximately 5 mg (0.6 cm diameter) pieces of Group 1 Set 1 film samples (theoretically containing ˜8 mU of enzyme) were added to wells. Next, buffer (50 mM MES, pH 6, 0.25% Triton X 100) and enzyme substrate (0.1 mM Resorufin Cellobioside) were added to wells, and the fluorescent signal was monitored for 30 to 50 minutes (with an excitation 550 nm, an emission of 590 nm). Unexpectedly, it was discovered that in-film enzymatic activity can be directly measured without initial “harsh extraction.” (FIG. 6B). Further, it was observed that higher PVC levels consistently led to higher in-film enzymatic activity.

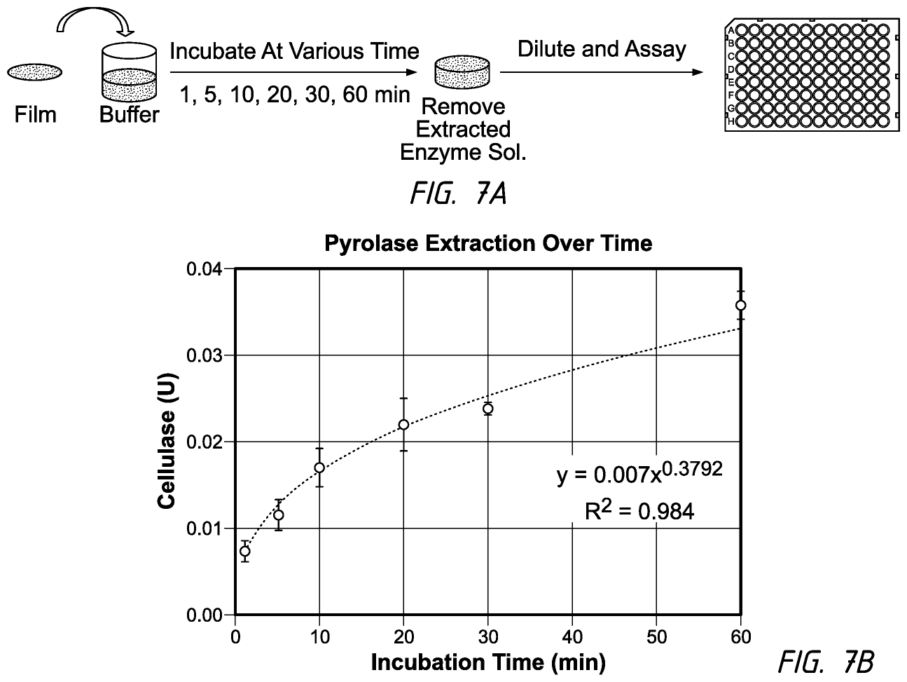

Next, a “soft” enzyme extraction from film sample using assay buffer under native conditions was developed. A 5.5 mg piece (0.6 cm diameter) of Group 1 Set 3 3B film sample (theoretically containing 47.8 U/g of enzyme) was used, and thus had a theoretical enzyme activity of 0.55 U per piece. As schematically depicted in FIG. 7A, the film was added to 200 μL of assay buffer (50 mM MES, pH 6, 0.25% Triton X 100) and incubated at room temperature for various times (1, 5, 10, 20, 30, and 60 minutes). The extracted enzyme solution was removed, diluted, and assayed according to the protocol described above. FIG. 7B shows the increase in cellulase extraction as the incubation time period increased.

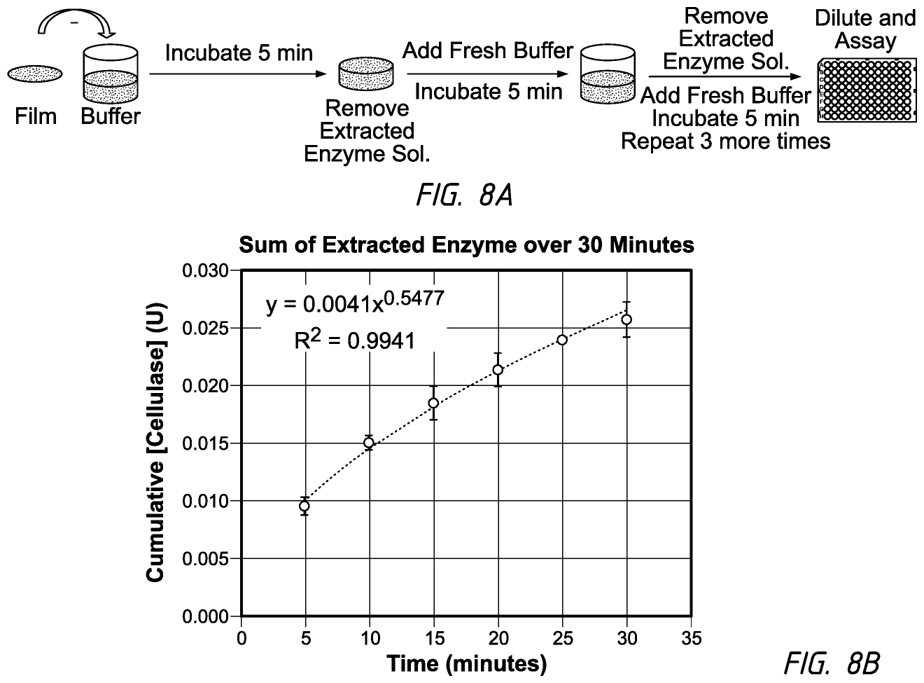

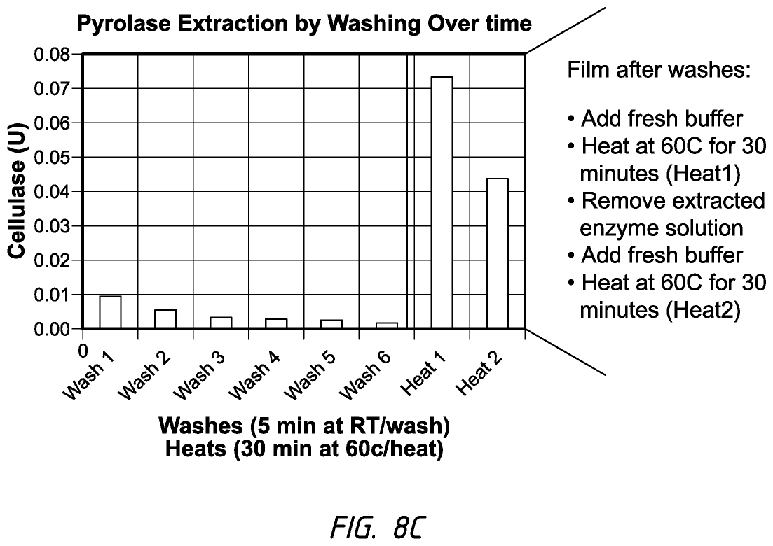

Studies were next conducted to determine the level of enzyme recovery from “soft” enzyme extraction as compared to “harsh” extraction. As schematically depicted in FIG. 8A, film was placed in the “soft” extraction buffer for five minutes at room temperature, the extracted enzyme solution was removed, fresh buffer was added, and this process was repeated several times. Extracted enzyme solutions were diluted and assayed as described above. FIG. 8B shows cumulative cellulase activity over 30 minutes with the six washes. Following six 5-minute washes at room temperature (Washes 1-6), fresh buffer was added to the film sample and incubated at 60° C. for 30 minutes (Heat 1). At the end of this incubation, the extracted enzyme solution was removed, fresh buffer was again added to the film sample, and a second incubation at 60° C. for 30 minutes was performed (Heat 2). Interestingly, it was found that there is low enzyme recovery from “soft” enzyme extraction as compared to “harsh” extraction (FIG. 8C).

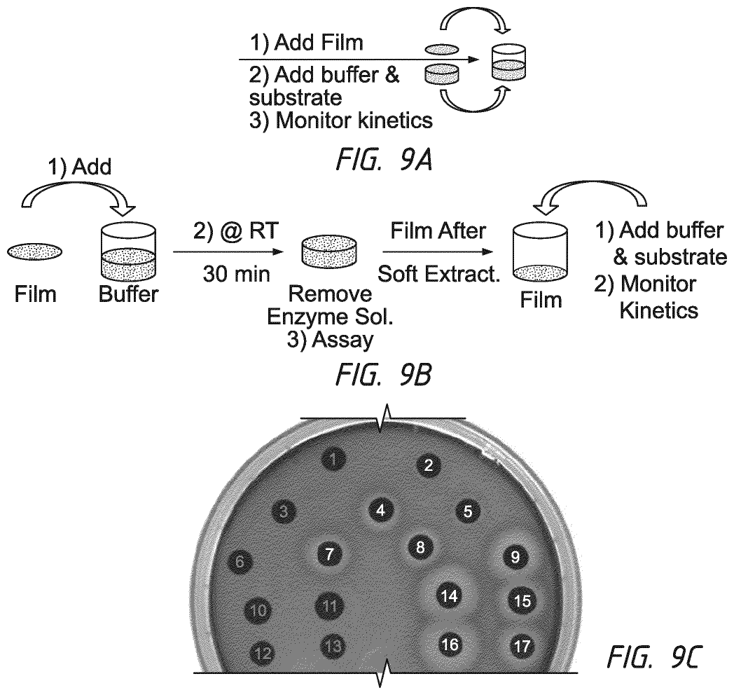

Based on the foregoing investigations, an in-film total assay, a soft extraction assay, and an in-film residual assay were developed for testing of the paint samples (schematically depicted in FIGS. 9A and 9B). The in-film total assay, soft extraction assay, and in-film residual assay each comprise a 30 minute incubation at room temperature in 50 mM MES Buffer with 0.25% Triton-X100, pH 6. However, the soft extraction assay includes assaying the enzyme activity of the extracted enzyme solution (comprising soluble protein), while the residual enzyme activity uses the film treated in the aforementioned “soft” extraction.

Development of an Assay for Visualizing in-Film Enzyme Activity

The aforementioned assay methods comprise, in some embodiments, biochemical analysis of film samples by spectrophotometric methods. To enable visualization of in-film enzyme activity, an agar plate-based method was developed. An agar plate containing 5% agar media and 0.1% Azo-Barley Glucan (a native substrate for cellulase/mannanase) was prepared. Dry film samples were placed on the surface of agar, with the bottom film surface in contact with the agar. The agar media took a base color due to the presence of the substrate. As shown in FIG. 9C, incubation with film samples loaded with 0.1% cellulase (but not control films without enzyme) caused a clearing zone to appear in the agar around and underneath film samples; this effect is due to the long chain carbohydrate being cleaved by the enzyme. Unexpectedly, this assay was found to work when the paint film in a semi-dry state and/or when there is no soaking of the film in solution, which can advantageously mimic application conditions. The limited number of steps, the low cost, and the straightforward visual analysis of this assay provides many advantages and potential applications, such as, for example, use as a quick screening tool of the relative activity of enzymes in a plurality of dry paint samples. Collectively, these studies provide proof of principle that multiple classes of enzymes directly embedded in wet paint retain their activity, and further that they remain active following film formation.

### Example 4: In-Film Activity of Paint Samples with Different Formulations

In this example, the in-film enzyme activity assay methods developed in Example 3 were employed to examine enzyme activity in dry paint films under native conditions. The paint samples described above were assayed using the in-film total assays, soft extraction assays, and in-film residual assays of Example 3 to elucidate the impact of different paint formulation components (e.g., PVC levels, filler chemistries) on in-film enzyme activity.

### In-Film Activity Analysis of Group 2 Paint Samples

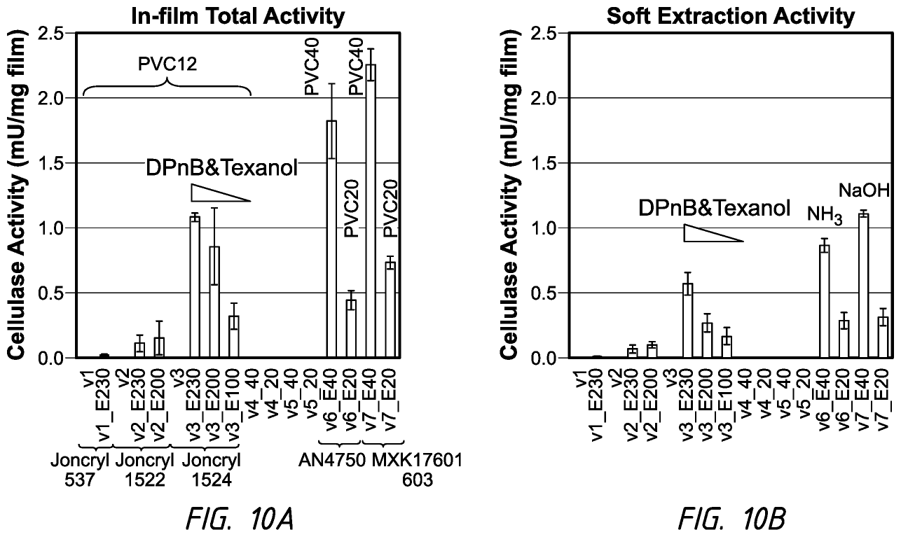

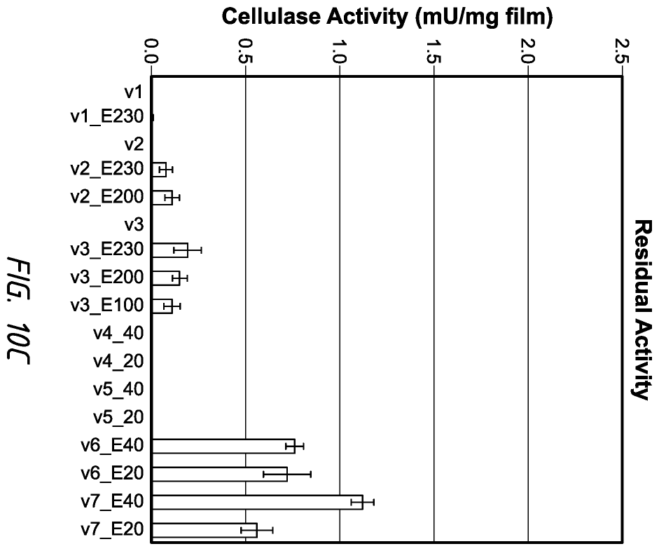

The in-film total activity, soft extraction activity, residual activity of cellulase detected in Group 2 dry film samples is shown in FIGS. 10A, 10B, and 10C, respectively. Based on an assumption that 100% activity is 31.2 mU/mg, less than 10% of enzyme activity was detected by the in-film assay. Latex type was discovered to have an impact on in-film activity, with films comprising Acronal® 4750 and MXK17601-603 exhibiting superior cellulase activity. Additionally, in-film enzyme activity increased with increasing levels of coalescents (DPnB and Texanol). Unexpectedly, higher PVC levels were found to result in higher in-film enzyme activity.

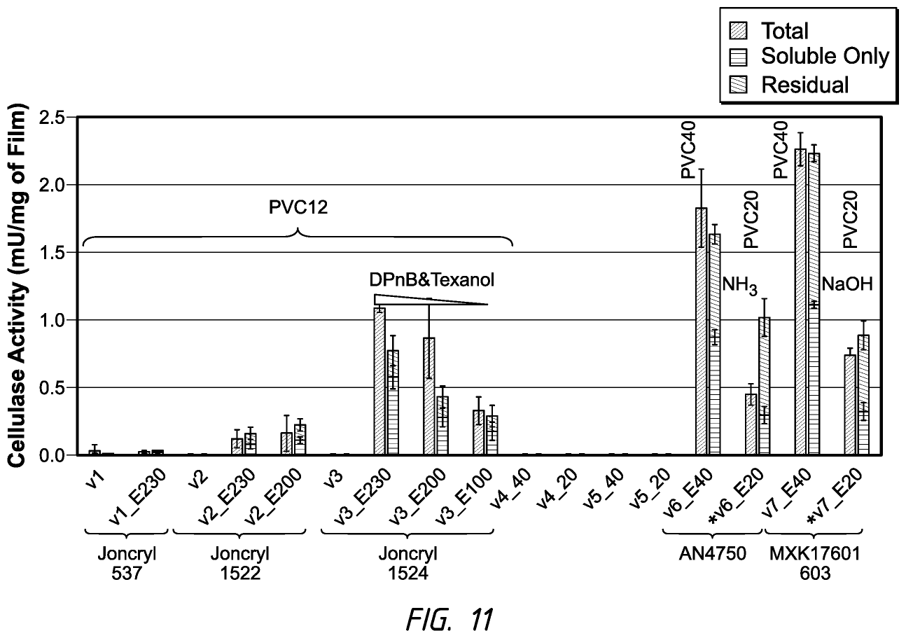

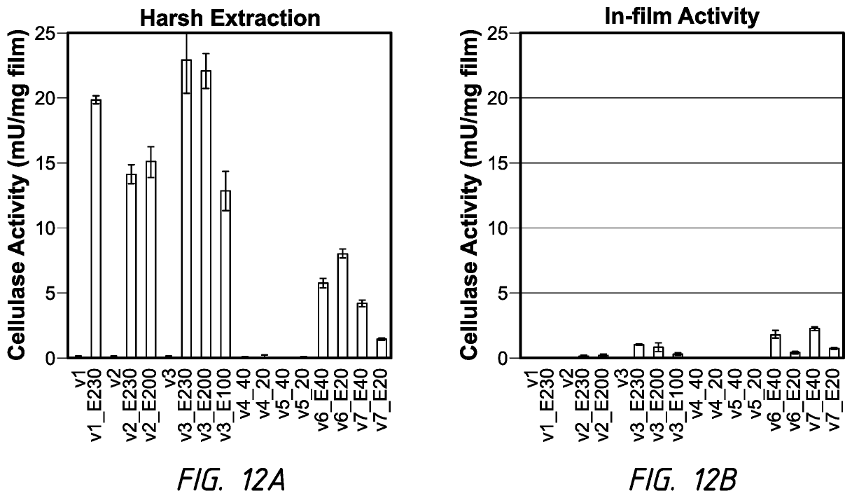

A set of experiments was performed to determine if the total in-film enzyme activity reflects the sum of soluble enzyme activity from “soft” extraction and residual enzyme activity in the film after soft extraction. As shown in FIG. 11, this hypothesis was confirmed. Interestingly, more than 50% of in-film activity was found to be derived from soluble enzyme (“soft” extraction). Next, studies were performed to elucidate the relationship between enzyme activity levels detected from in-film assays and enzyme activity levels detected from “harsh” extraction of dry film samples. As seen in FIG. 12, enzyme activity levels detected using in-film assays are only a small fraction of the enzyme activity detected following “harsh” extraction. Table 4 depicts percent in-film activity (Activity_{in-film}/Activity_{Harsh Extraction}) values calculated from the experiments depicted in FIG. 12, which is an activity comparison, not protein quantification. Interestingly, higher “harsh” extractability was not found to correlate with higher in-film activity. Our studies show that a number of paint formulation components, including DPnB levels, Texanol levels, PVC, neutralizer type, and latex type all contribute to enzyme extractability and in-film activity.

TABLE 4
PERCENT IN-FILM ACTIVITY

| | % In-film |
|---|---|
| Samples | Activity * |
| v1_E230 | 0.15 |
| v2_E230 | 0.84 |
| v2_E200 | 1.05 |
| v3_E230 | 4.75 |
| v3_E200 | 3.93 |
| v3_E100 | 2.56 |
| v6_E40 | 31.92 |
| v6_E20 | 5.61 |
| v7_E40 | 54.06 |
| v7_E20 | 51.31 |

* Activity comparison, not protein quantification

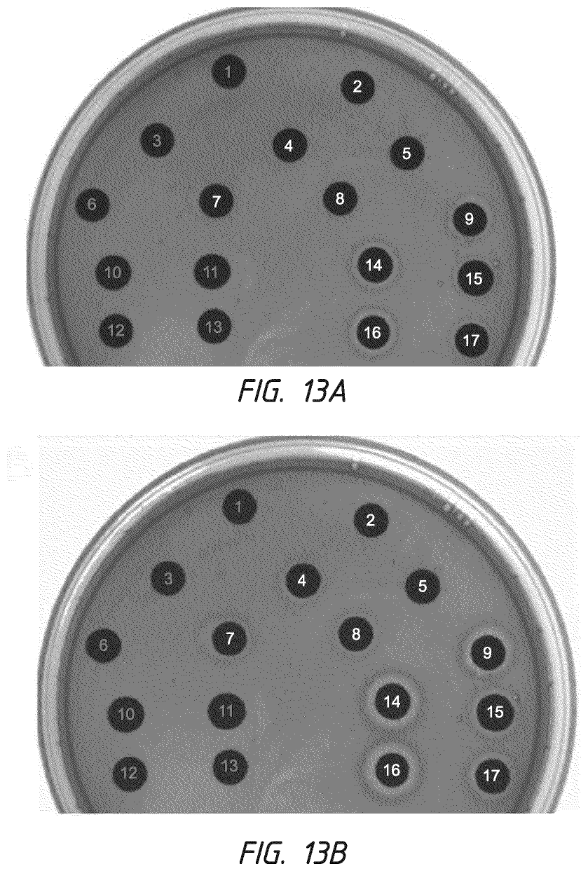

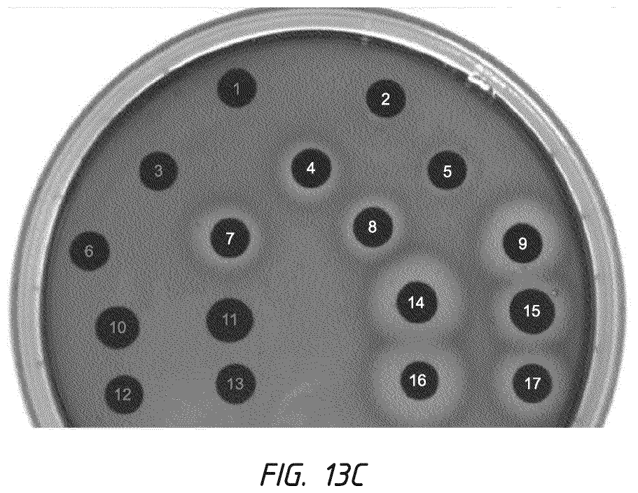

To confirm the results of the biochemical studies above, the in-film enzyme activity of Group 2 dry film samples was visualized using the agar plate method developed in Example 3. The paint formulations indicated in Table 1 were loaded with 0.1% cellulase (samples 2, 4, 5, 7, 8, 9, 14, 15, 16, 17); parallel film samples (1, 3, 6, 10, 11, 12, 13) not loaded with enzyme were used as controls. Plates incubated for 3, 7, and 22 at 37° C. showed a progressive increase in zone of clearing around films containing enzymes (FIGS. 13A, 13B, and 13C, respectively). Importantly, these qualitative visual results are consistent with the in-film activity biochemical assay, including the discovery that increasing PVC levels increases in-film enzyme activity.

TABLE 5
PAINT FORMULATIONS ASSAYED USING THE AGAR PLATE
METHOD

| | Sample | | | | | | | | | | |
|---|---|---|---|---|---|---|---|---|---|---|---|
| | 2 | 4 | 5 | 7 | 8 | 9 | Sample | 14 | 15 | 16 | 17 |
| Name | v1_E | v2_E | v2_E | v3_E | v3_E | v3_E | Name | v6_E | v6_E | v7_E4 | v7_E2 |
| | 230 | 230 | 200 | 230 | 200 | 100 | | 40 | 20 | 0 | 0 |
| Latex | Joncryl | Joncryl | Joncryl | Joncryl | Joncryl | Joncryl | Latex | AN4 | AN4 | MXK17 | MXK17 |
| | 537 | 1522 | 1522 | 1524 | 1524 | 1524 | | 750 | 750 | 601MX | 601MX |
| | | | | | | | | | | K1760 | K1760 |
| | | | | | | | | | | 1-603 | 1-603 |
| Neutralizer | | | | | | | Neutralizer | Ammonia | Ammonia | NaOH | NaOH |
| | | | | | | | Kronos | 100 | 48.8 | 100 | 48.8 |
| | | | | | | | 2310 | | | | |
| | | | | | | | Duramite | 100 | 48.8 | 100 | 48.8 |
| Dipropylene | 60 | 60 | 50 | 60 | 50 | 22 | | | | | |
| glycol | | | | | | | | | | | |
| n-butyl ether | | | | | | | | | | | |
| (DPnB) | | | | | | | | | | | |
| Texanol | 30 | 30 | 25 | 30 | 25 | 11 | | | | | |
| PVC | 12 | 12 | 12 | 12 | 12 | 12 | PVC | 40% | 20% | 40% | 20% |

### In-Film Activity Analysis of Group 1 Set 1 Paint Samples

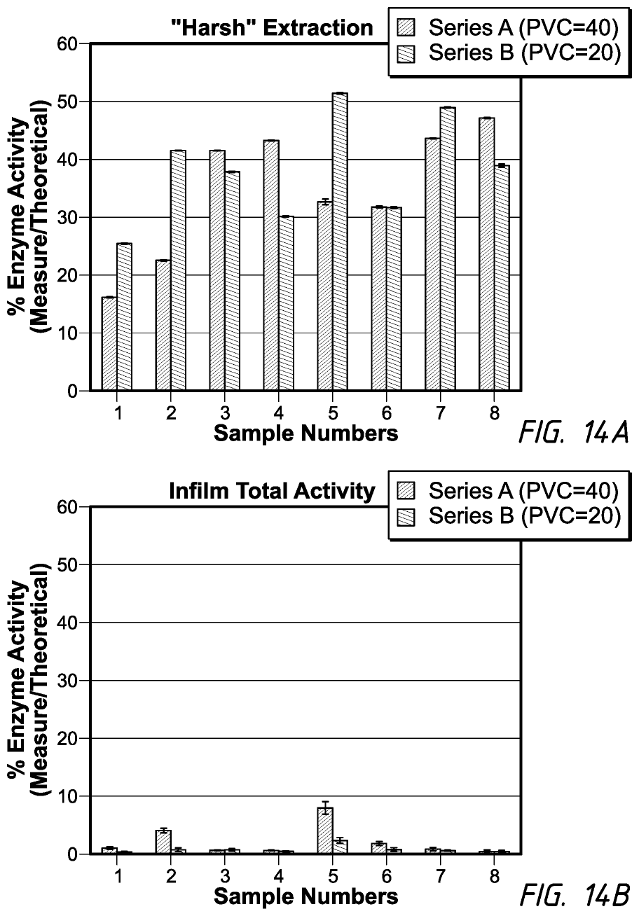

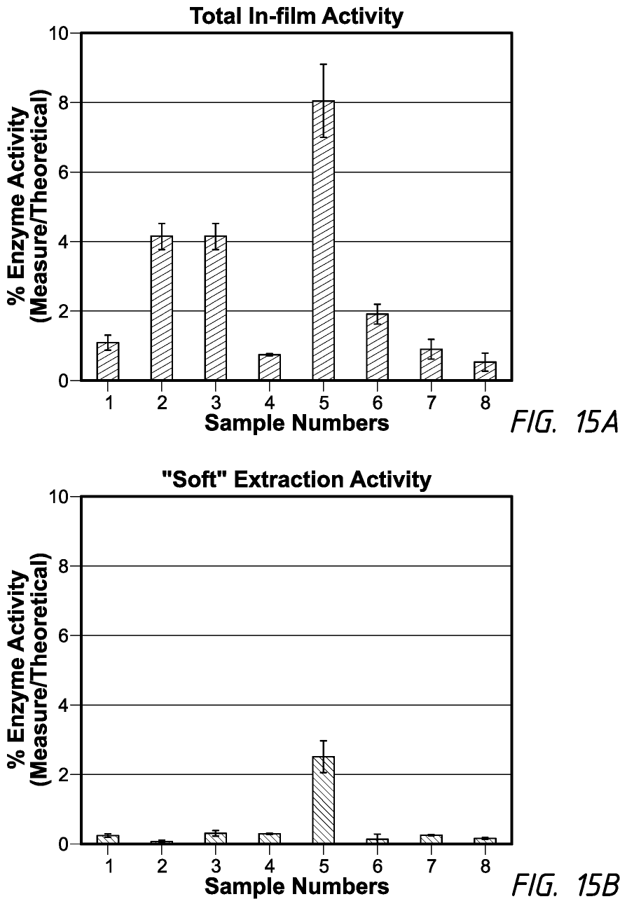

FIGS. 14A and 14B show enzyme activity detected by “harsh” extraction and total in-film activity assays of Group 1 Set 1 dry film samples, respectively. Consistent with the results of other experiments herein, higher in-film activity was detected from higher PVC samples; furthermore, consistent with the above results, this effect was not observed with “harsh” extraction. Less than 10% of enzyme activity was found to be detected by in-film assay. Sample 5 (comprising a SiO₂—Celatom filler) exhibited the highest in-film activity. These data provide further evidence that increasing PVC levels increases in-film enzyme activity. And consistent with results described above, higher “harsh” extractability does not correlate with higher in-film activity. Group 1 Set 1A dry film samples, which have a 40% PVC value, were further assayed by in-film total activity assays and “soft” extraction assays (FIG. 15). Roughly 25% of total in-film activity was found to be derived from soluble enzyme.

### In-Film Activity Analysis of Group 1 Set 3 Paint Samples

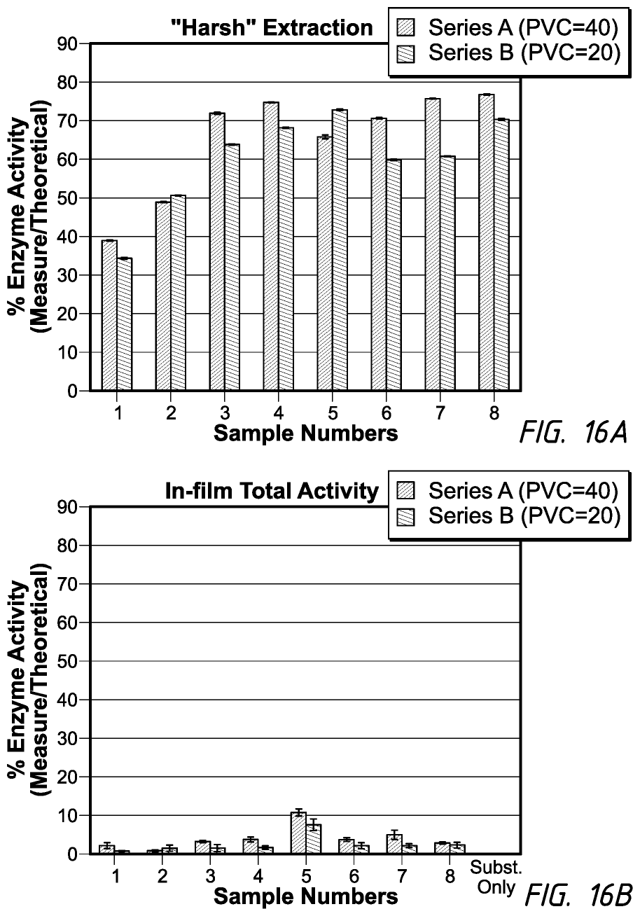

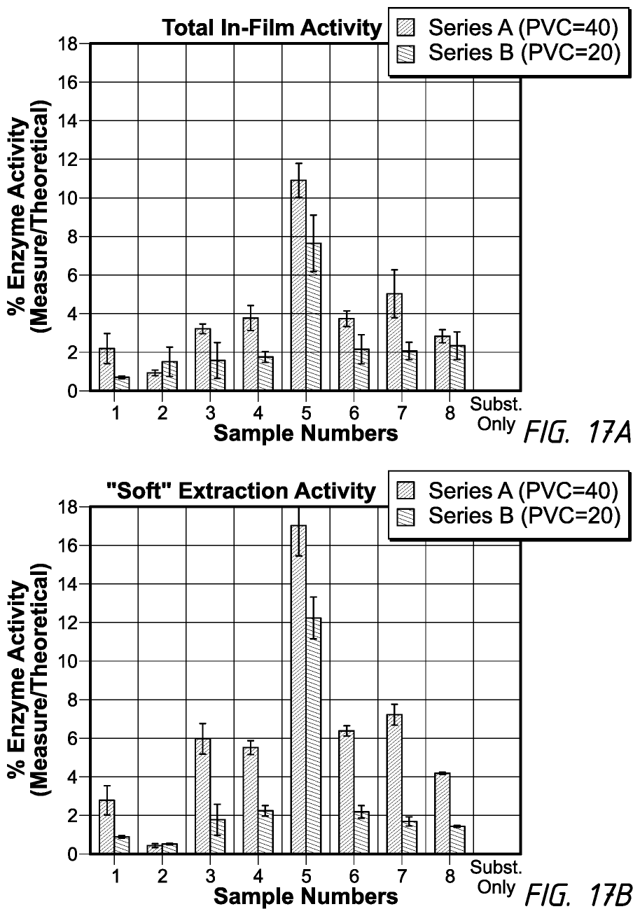

Group 1 Set 3 dry film samples were analyzed by “harsh” extraction and total in-film activity assays (FIGS. 16A and 16B, respectively). The Set 3 dry film samples comprise a higher loading of enzyme (˜96 U/g) as compared to the Set 1 dry film samples assayed above (˜1.8 U/g). Consistent with the results of other experiments herein, higher in-film activity was detected from higher PVC samples (and this effect was not observed with “harsh” extraction). Less than 12% of enzyme activity was found to be detected by in-film assay. Consistent with the results above, Sample 5 (comprising SiO₂—Celatom filler) exhibited the highest in-film activity. These data provide further evidence that increasing PVC levels increases in-film enzyme activity. And consistent with results described above, higher “harsh” extractability does not correlate with higher in-film activity. Group 1 Set 3 dry film samples were further analyzed by in-film total activity assays and “soft” extraction assays (FIG. 17). As seen with the Set 1 samples, the majority of the in-film activity comes from “soft” extraction.

The experiments described herein yielded a number of insights regarding the in-film enzyme activity by the assays developed as well as elucidate the influence of paint formulation components on in-film enzyme activity. In-film enzyme activity was found to be significantly lower than enzyme recovery from “harsh” extraction, and higher activity from “harsh” extraction does not correlate with higher in-from activity. The enzyme activity from in-film assay is significantly lower than that of “free” enzyme in solution at the theoretical inclusion level, with values of only about 10% or lower observed. The reason is unlikely due to irreversible enzyme inactivation in films, as shown by the results that the enzyme recovered from “harsh” extraction remain highly active. It is therefore reasonable to conclude that the enzyme remains active in films with reduced specific activity. This is possibly due to multiple factors that restrict enzyme catalytic conversion rate in-film, including diffusion of substrate and/or enzyme, substrate accessibility to enzyme, and enzyme conformation in film matrix. The mass balance of the total in-film activity was found to be roughly the sum of that of “free” enzyme that can be extracted by “soft” extraction and the residual activity remaining in the film. Finally, multiple formulation components were unexpectedly found to have a pronounced effect on in-film enzyme activity. Higher levels of coalescing agents were found to increase in-film activity. Paint formulations with higher PVC levels also demonstrated increased in-film activity. Additionally, latex type and filler type both impacted in-film activity, with formulations comprising SiO₂ (Celatom) exhibiting the highest activity. Importantly, these results were confirmed with the use of different types of assays as well as different types of paint formulations. Collectively, these studies provide further proof of principle that multiple classes of enzymes directly embedded in wet paint retain their activity, and further that they remain active following film formation.

### Example 5: Design of New in-Film Activity Assays for the Proof-of-Concept Analyses of Other Enzymes Classes

This example shows that other classes of enzymes directly embedded in wet paint retain their activity following film formation. Another aim of the present set of experiments was to develop biochemical assay and agar plate protocols that can reliably and accurately determine enzyme activity directly in-film for an expanded class of enzymes, including amylases, lipases, proteases, laccases, ureases. Agar plate screening is a rapid and efficient technique to visualize and screen enzyme activity. Finally, studies elucidating the impact of different paint formulation components (e.g., PVC levels, filler chemistries) on in-film enzyme activity for these enzyme classes were also undertaken.

Agar Plate in-Film Activity Assays

Protocol

Agar plates prepared consisted of 2% Difco Agar Noble and an enzyme's substrate. The substrate was selected so that after the enzymatic conversion of the substrate to the product, a color change could be visually observed. The color change can come from the substrate or product itself, or from a contrasting agent co-imbedded in the agar with the substrate. To achieve homogeneity, a 2% Difco Agar Noble solution is boiled to a molten solution and cooled down on benchtop to ˜60° C. before addition of enzyme's substrates as follows:

Laccase substrate: 0.2 mM substrate (syringaldazine)

Lipase substrate: 1% Vegetable oil; 2% Nile Red

Amylase substrate: 0.7% red starch

Protease substrate: 0.5% Non-fat dried milk

The mixture was then poured to a media plate and cooled to room temperature to allow solidification.

Pieces of dry enzyme-containing paint films (e.g., a 0.6-cm in diameter circular piece cut by a hole puncher) was placed on top of the agar surface. The moisture from the agar partially wets the film, allowing the substrate to migrate to the paint film and allowing the enzyme from the film to migrate to the immediate adjacent area in the agar. Upon the conversion of the substrate to the product by the enzyme in the agar, a color change (increase in intensity, decrease in intensity, disappearance or appearance of color) can be visually observed and the image can be captured by an imager or camera.

Results

Amylases, lipases, proteases, and laccases were embedded in paint formulations equivalent to Group 1 Sample 7A/B (comprising Minex 4 filler [(NaK)Al₂(AlSi₃)O₁₀(OH)₂]).

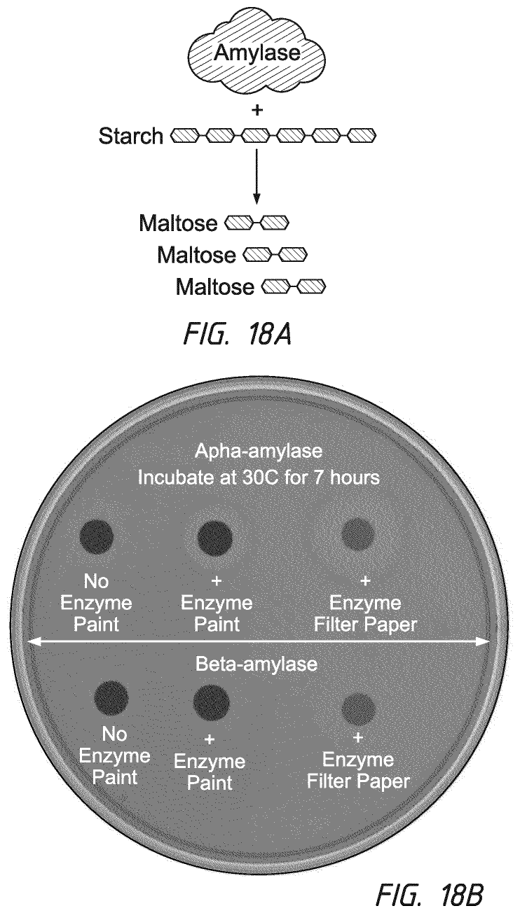

A red starch agar plate was prepared comprising 5% agar and 0.7% red starch. FIG. 18A shows a schematic representation of the enzyme-catalyzed reaction underlying the red starch agar plate assay according to several embodiments disclosed herein. Dry paint film samples comprising 0.1% alpha-amylase or 0.1% beta-amylase were incubated on the surface of the agar at 30° C. for 7 hours. Filter paper loaded with amylase and paint film not loaded with amylase serve as positive and negative controls, respectively. As shown in FIG. 18B, the dry film sample loaded with alpha amylase (endo and exo acting) showed good starch digestion (indicated by a zone of clearing) while beta amylase (exo acting only) showed poor starch digestion.

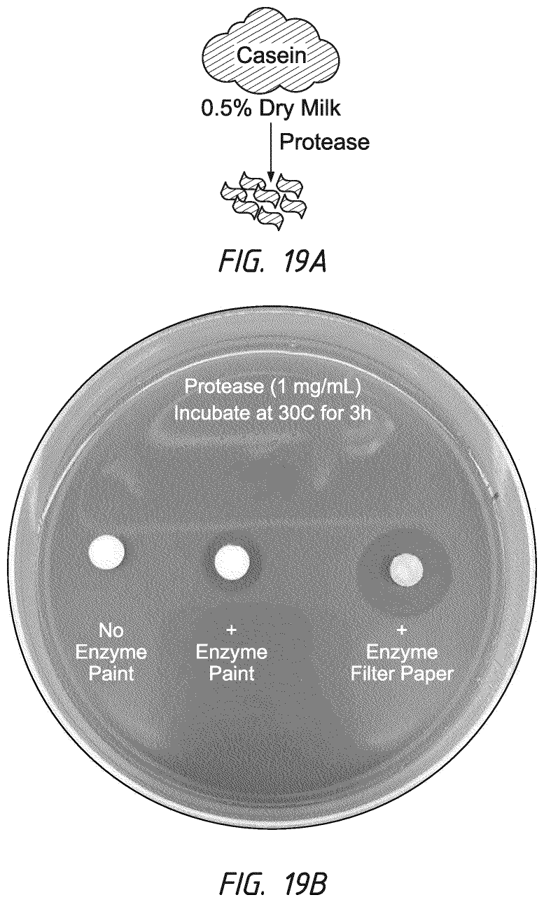

A milk agar plate was prepared comprising 2% agar and 0.5% non-fat dried milk (in some embodiments a blue dye was added for enhancing contrast). FIG. 19A shows a schematic representation of the enzyme-catalyzed reaction underlying the milk agar plate assay according to several embodiments disclosed herein. Dry paint film samples comprising 0.1% (1 mg/mL) protease were incubated on the surface of the agar at 30° C. for 3 hours. Filter paper loaded with protease and paint film not loaded with protease serve as positive and negative controls, respectively. As shown in FIG. 19B, the dry film sample loaded with acetyl lysine protease exhibited a zone of altered contrast surrounding the film.

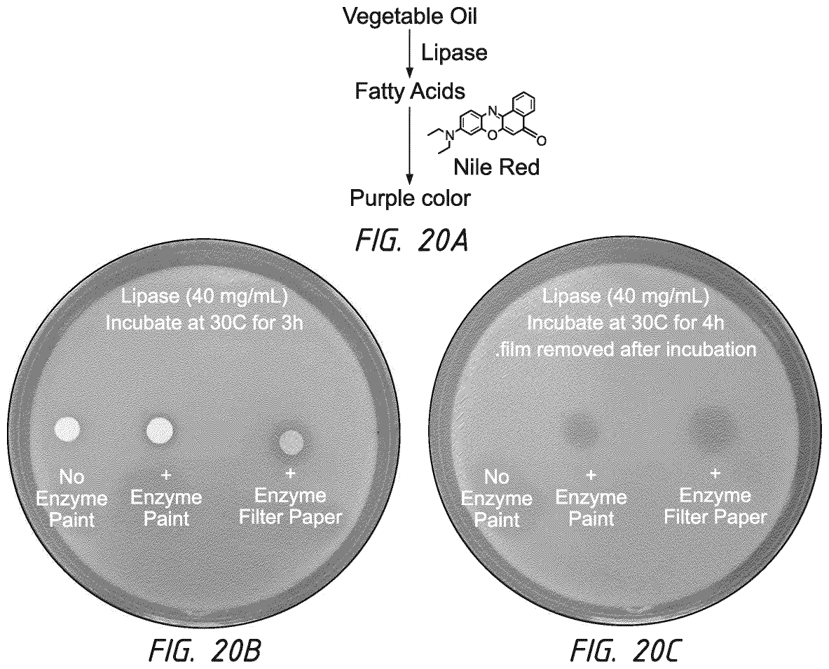

A vegetable oil agar plate was prepared comprising 2% agar, 1% vegetable oil, and 2% Nile Red. FIG. 20A shows a schematic representation of the enzyme-catalyzed reaction underlying the vegetable oil agar plate assay according to several embodiments disclosed herein. Dry paint film samples comprising 4% (40 mg/mL) lipase were incubated on the surface of the agar at 30° C. for 3 hours. Filter paper loaded with lipase and paint film not loaded with lipase serve as positive and negative controls, respectively. As shown in FIG. 20B, the dry film sample loaded with lipase exhibited a red zone surrounding the film. This plate was incubated for an additional hour and the film was removed, which revealed that the red shift in the color of the agar also occurred beneath the film (FIG. 20C).

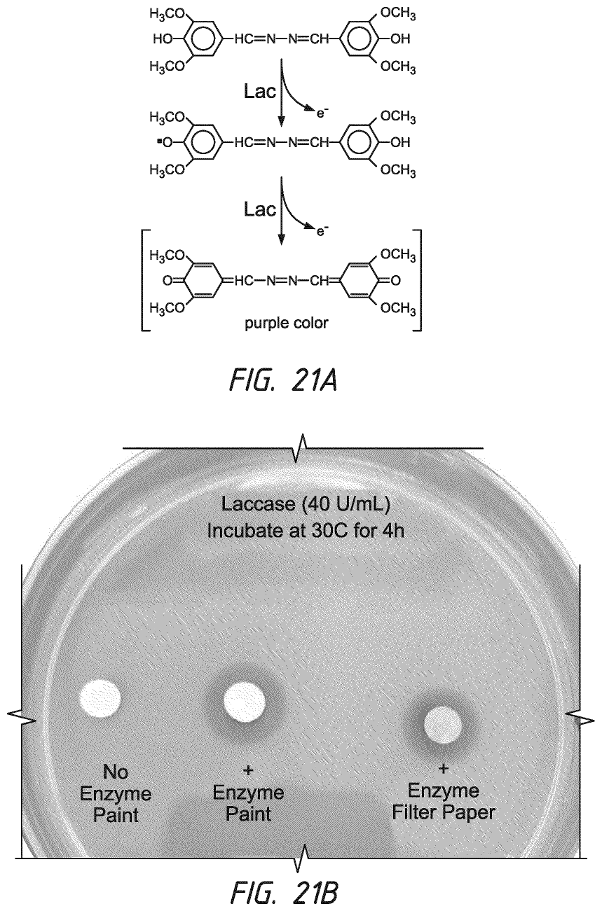

A syringaldazine (SGZ) agar plate was prepared comprising 2% agar and 0.2 mM SGZ. FIG. 21A shows a schematic representation of the enzyme-catalyzed reaction underlying the SGZ plate assay according to several embodiments disclosed herein. Dry paint film samples comprising 40 U/mL laccase were incubated on the surface of the agar at 30° C. for 4 hours. Filter paper loaded with laccase and paint film not loaded with laccase serve as positive and negative controls, respectively. As shown in FIG. 21B, the dry film sample loaded with laccase (a polyphenol oxidase) exhibited a purple zone surrounding the film owing to its ability to oxidize phenolic compounds.

These agar plate studies provide proof of concept that amylases, lipases, proteases, and laccases can directly embedded in wet paint and retain their activity in-film following film formation. Further, these experiments indicate that the agar plate assays that can reliably and accurately determine the activity of amylases, lipases, proteases, and laccases directly in-film. Given the significant impact of PVC levels on in-film cellulase activity of cellulase that we observed, agar plate assays investigating the impact of PVC levels and filler type on the in-film activity of amylases, lipases, proteases, and laccases were undertaken. Table 6 depicts the paint formulations for these classes of enzymes. The incorporations levels of amylase, protease, laccase, and lipase were 1%, 0.1%, 41.2 U/mL, and 0.1%, respectively. Dry film contains twice as much film due to solvent evaluation; thus, 0.01% in wet paint implies 0.02% in dry film.

TABLE 6
PAINT FORMULATIONS FOR TESTING
MULTIPLE ENZYME CLASSES

| Raw Materials | Description | 0A | 0B | 0C | 0D |
|---|---|---|---|---|---|
| Filler Type | | Minex 4 | Minex 4 | Celatom | Celatom |
| Water | Solvent | 180.7 | 123 | 180.7 | 123 |
| Ammonia | Neutralizing | 0.7 | 0.7 | 0.7 | 0.7 |
| | amine | | | | |
| Dispex CX 4340 | Dispersing | 4 | 4.6 | 4 | 4.6 |
| | agent | | | | |
| Foamstar ST 2420 | Defoamer | 2 | 2.3 | 2 | 2.3 |
| Proxel DB 20 | Biocide | 3 | 3.5 | 3 | 3.5 |
| Kronos 2310 | TiO2 | 200 | 97.7 | 200 | 97.7 |
| | pigment | | | | |
| Filler | Filler | 200 | 97.7 | 200 | 97.7 |
| Attagel 50 | Thickener | 4 | 4.6 | 4 | 4.6 |
| Mix for 10-15 min, | | | | | |
| then add: | | | | | |
| Water | Solvent | 75 | 57.5 | 75 | 57.5 |
| Foamstar ST 2420 | Defoamer | 2 | 2.3 | 2 | 2.3 |
| Hydropalat WE 3320 | Wetting | 2 | 2.3 | 2 | 2.3 |
| | agent | | | | |
| Loxanol CA 5320 | Coalescing | 9 | 10.3 | 9 | 10.3 |
| | agent | | | | |
| Acronal 4750 | Binder | 425 | 564.9 | 425 | 564.9 |
| Rheovis PE 1331 | Rheology | 12.5 | 14.4 | 12.5 | 14.4 |
| | modifier | | | | |
| Rheovis PU 1191 | Rheology | 1 | 1.2 | 1 | 1.2 |
| | modifier | | | | |
| Total grams (equiv. | | 1120.9 | 986.8 | 1120.9 | 986.8 |
| to lbs/100 gal) | | | | | |

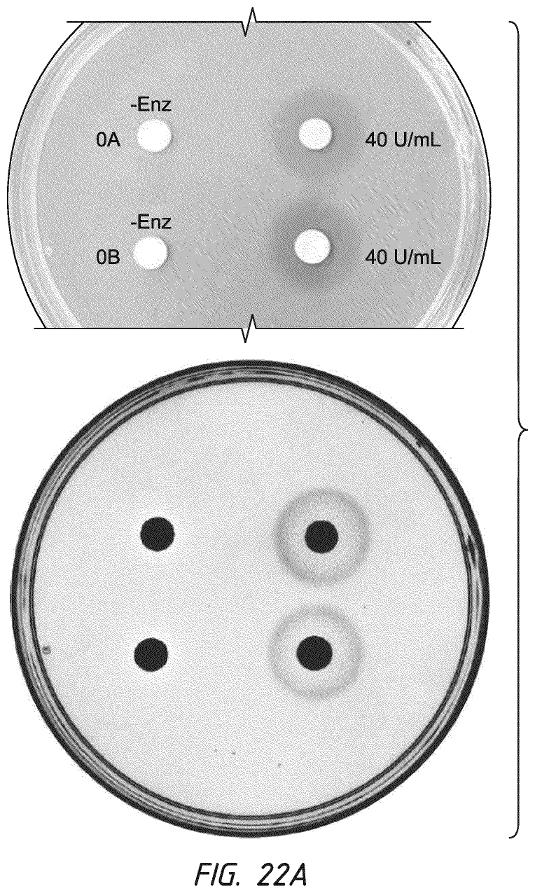

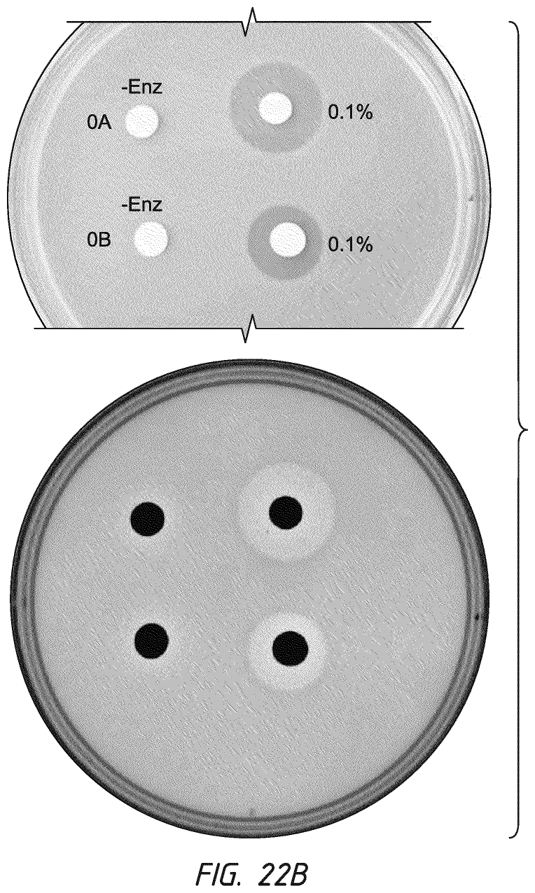

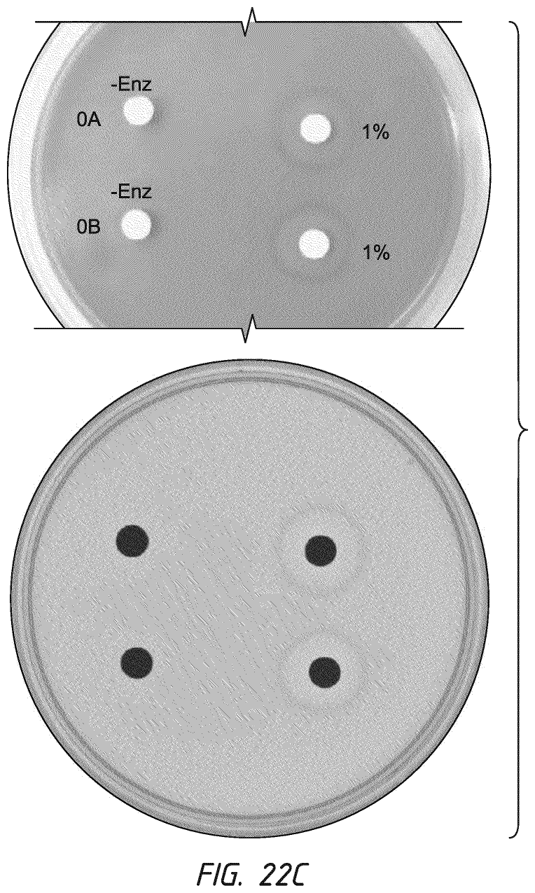

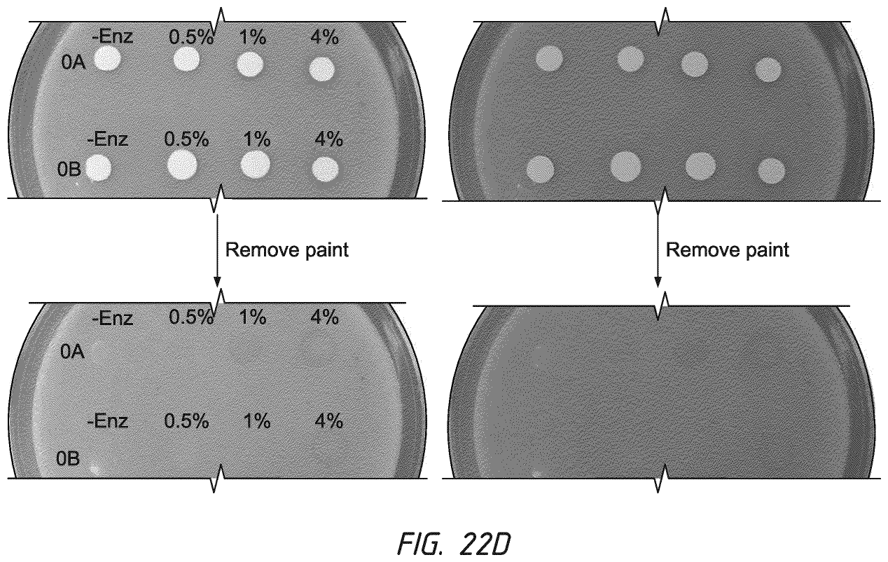

Laccase, protease, alpha-amylase, and lipase were added to paint formulations comprising a Minex 4 filler and a PVC of either 40% (0A samples) or 20% (0B samples). FIG. 22 shows that the positive impact of PVC levels on in-film enzyme activity is also readily apparent with films comprising lipases and proteases.

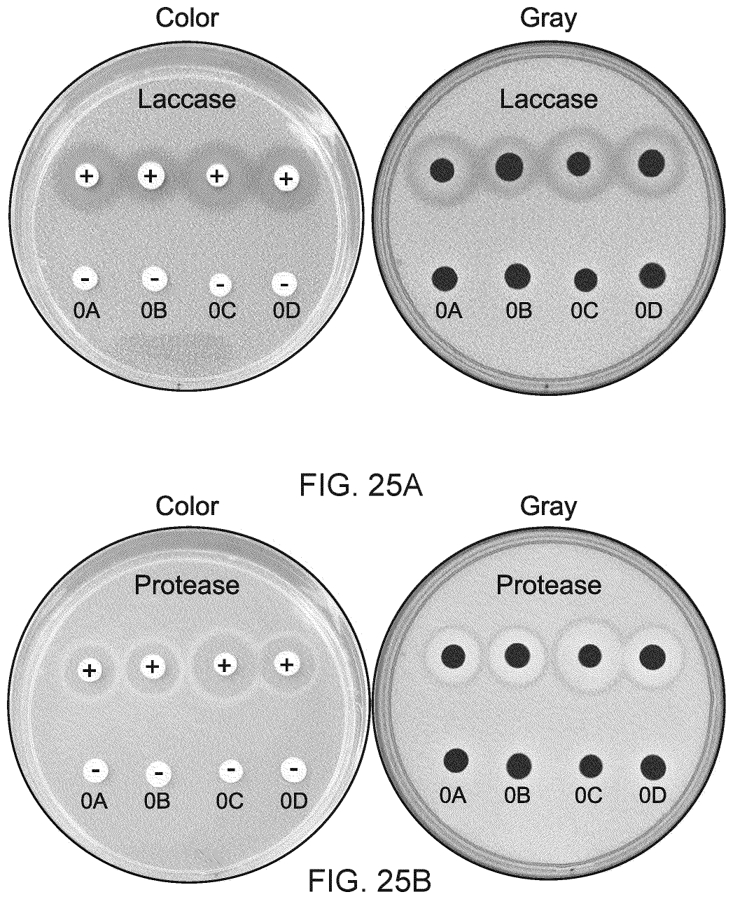

Additionally, paint formulations comprising either Minex 4 filler (0A and 0B samples in Table 6) or Celatom filler (0C and 0D samples) and a PVC of either 40% (0A and 0C samples) or 20% (0B and 0D samples) were embedded with laccase and protease, and the films were assayed via agar plate. Both enzyme classes exhibited higher in-film activity in paints formulated with Celatom as the filler than Minex 4 (FIG. 25). In-film activity was found to increase with higher PVC level for all laccase samples tested as well as for proteases embedded in Celatom-containing paint; however, this trend was not observed with protease embedded in Minex-containing paint.

Biochemical in-Film Activity Assays

Problem & Solution for Biochemical in-Film Activity Assay

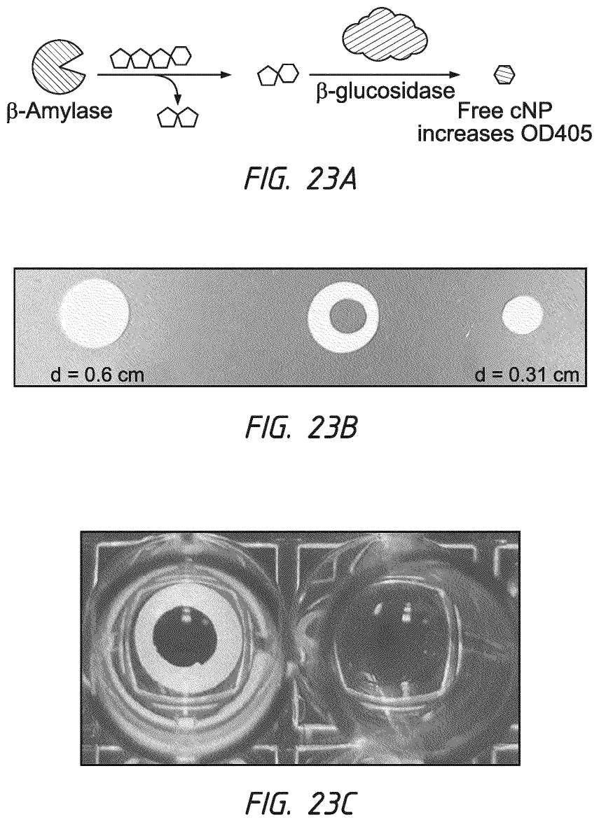

Colorimetric assays are convenient and fast in-vitro assays that evaluate enzyme activity based on the change in absorbance at a specific wavelength of a substrate upon interacting with an enzyme. This assay requires an incident light path to pass through a testing solution and records the absorbance of that light. In the case analyzing enzyme activity in dry paint films, measurement of absorbance is not feasible as the dry paint film blocks the light path. This light blockage can cause a number of issues depending on the enzyme, paint, and substrate being tested, including: 1) an inaccurate readout of enzyme activity; 2) an extended assay period required; 3) higher levels of substrate and/or enzyme required; and/or 4) incompatibility of particular paint formulations with the assay. This challenge is particular problematic as significant screening can be required to elucidate the optimal paint formulation for a given enzyme and/or contemplated paint application. Provided herein is a solution to this problem: configuring the film to allow the incident light path to pass through, such as, for example, by removing an interior portion of the film before it is placed in the sample well. In some embodiments, this method comprises cutting out the middle part of the film to allow light to pass through as shown in FIG. 23B). Importantly, as shown in FIG. 23C, this solution is compatible with the assay of films in a 96-well plate. In some embodiments, dry paint films containing enzymes are cut into an “O-ring shape” pieces using two different sizes of hole punchers. First, a bigger hole puncher with a 0.6 cm diameter cuts the dry paint film into a circular piece that fits perfectly into a well of a 96-well plate. This circular piece of dry paint film is further cut with a smaller hole puncher (diameter=0.31 cm) at the center. This creates a 0.6 cm disk with a 0.31 cm hollow center (or “O-ring shape”). As a result, the hollow center allows the incident light to pass through the center of each well and enable recording of the absorbance of that light, while enzymes in “O-ring” portion of the paint film can interact with the substrate solution added to the well. Importantly, this method allows detection of enzyme activity from enzyme that was released into the solution from the paint film as well as immobilized enzyme in the dry paint film. In addition, it facilitates the evaluation of enzyme in different dry paint film using a microtiter plates in a high throughput manner. The dimensions described here are fitted for 96-well microtiter plate format; the sizes can be adjusted for other plate or non-plate formats for absorbance measurement.

Colorimetric Assays Procedures

5 mg of O-ring shape dry paint film containing an enzyme (laccase, lipase, protease, or amylase) was prepared using 2 different sizes of hole punchers (out diameter=0.6 cm, inner diameter=0.31 cm) and placed in a well of a 96 well plate. The activity assay conditions were as follows:

Laccase: 200 μL of 100 mM potassium phosphate buffer (pH 6.5) that contains 0.02 mM substrate (syringaldazine) was added to the well. Change in absorbance at 530 nm over time was recorded to determine the activity of laccase.

Lipase: 200 μL of 50 mM HEPES buffer (pH 7.5) that contains 100 mM NaCl, 20 mM CaCl2), 0.01% Triton-X100 and 1 mM substrate (4-nitrophenyl octanoate) was added to the well. Change in absorbance at 405 nm over time was recorded to determine the activity of lipase.

Amylase: 200 μL of 50 mM HEPES buffer (pH 7.5) that contains 0.1 mg/mL BSA, 1 U/mL of δ-glucosidase, and 4 mM substrate (2-chloro-4-nitrophenyl-β-D-maltotrioside) was added to the well. Change in absorbance at 405 nm over time was recorded to determine the activity of amylase. FIG. 23A shows the schematic representation of the colorimetric in-film amylase activity assay.

Protease: 200 μL of 50 mM HEPES buffer (pH 7.5) that contains 1 mM substrate (Succinyl-Ala-Ala-Pro-Phe-p-nitroanilide) was added to the well. Change in absorbance at 405 nm over time was recorded to determine the activity of protease.

Urease: 100 μL of 10 mM Phosphate buffer (pH 7.0) that contains 10 μL of substrate (urea solution provided with Urease Assay Kit from Sigma Aldrich) was added to dry paint film in a 96-well plate and incubated for 10 minutes. During this time, urease from paint converts urea into ammonia and carbon dioxide. 150 μL of detecting agents (Reagent A and Regent B provided with Urease Assay Kit from Sigma Aldrich) were then added to the solution. These reagents inhibit urease activity and allow ammonia to react with detecting agents to generate a blue color (wavelength is between 600-700 nm). Absorbance at 600-700 nm was recorded and compared to a urease standard curve to determine the activity of urease.

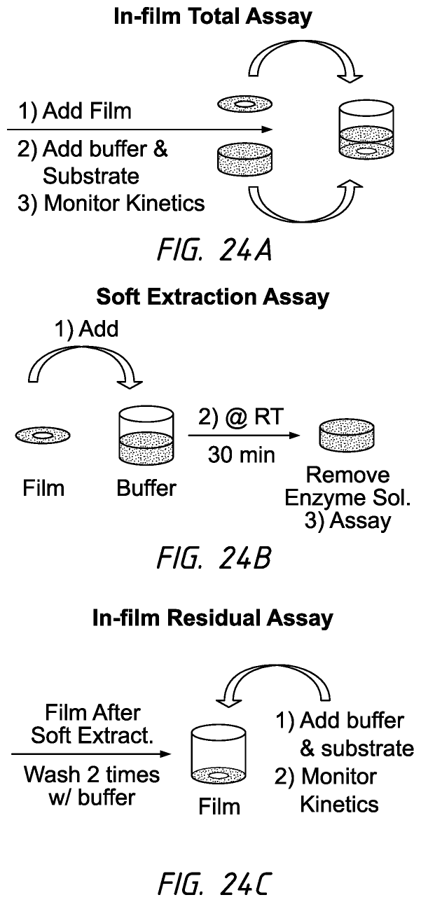

The total in-film activity assay comprises incubating the film with the assay buffer at room temperature for 30 minutes and measuring activity. The “soft” extraction activity assay comprises incubating the film in assay buffer for 30 minutes, removing the film, and measuring the activity of the soluble protein. The in-film residual assay comprises washing the film from the “soft” extraction assay in buffer and measuring the residual enzyme activity in the film. FIG. 24 shows schematic representations of an in-film total assay, a soft extraction assay and an in-film residual assay (FIGS. 24A, 24B, and 24C, respectively) according to several embodiments disclosed herein.

Results

Amylase (20 mg/g), lipase (2 mg/g), protease (0.2 mg/g), and laccase (82 U/g) were embedded in paint formulations in Table 6, comprising either Minex 4 filler (0A and 0B samples) or Celatom filler (0C and 0D samples) and a PVC of either 40% (0A and 0C samples) or 20% (0B and 0D samples). The film samples were assayed for total in-film, “soft” extraction and in-film residual activities.

Urease (4 U/g) was embedded in paint formulations equivalent to Group 2 Sample v7_E40/E20 (depicted in Table 7) which comprises the filler Duramite (CaCO₃) and comprises NaOH as the neutralizing agent.

TABLE 7
PAINT FORMULATIONS EMBEDDED WITH UREASE

| Formulation Name | | v5_40 | v5_20 |
|---|---|---|---|
| Latex | | MXK17601-603 | MXK17601-603 |
| Neutralizer | | NaOH (29%) | NaOH (29%) |
| Water | Solvent | 90.4 | 61.5 |
| Neutalizer | Neutralizing | 0.75 | 0.86 |
| | amine | | |
| Dispex CX 4340 | Dispersing | 2 | 2.3 |
| | agent | | |
| Foamstar ST 2420 | Defoamer | 1 | 1.15 |
| Proxel DB 20 | Biocide | 1.5 | 1.73 |
| Kronos 2310 | TiO2 pigment | 100 | 48.8 |
| Duramite | Filler | 100 | 48.8 |
| Attagel 50 | Thickener | 2 | 2.3 |
| Total | | 297.6 | 167.5 |
| Mix for 10-15 min, | | | |
| then add: | | | |
| Water | Solvent | 37.5 | 28.7 |
| Foamstar ST 2420 | Defoamer | 1 | 1.15 |
| Hydropalat WE 3320 | Wetting agent | 1 | 1.15 |
| Loxanol CA 5320 | Coalescing | 4.5 | 5.17 |
| | agent | | |
| Binder | Binder | 212.5 | 282.5 |
| Rheovis PE 1331 | Rheology | 6.25 | 7.18 |
| | modifier | | |
| Rheovis PU 1191 | Rheology | 0.5 | 0.58 |
| | modifier | | |
| Total grams (equiv. | | 560.9 | 493.9 |
| to lbs/100 gal) | | | |
| Viscosity target | KU | 95-100 | 95-100 |
| Viscosity target | ICI | 1.0-1.5 | 1.0-1.5 |
| Volume Solids | | 39% | 39% |
| PVC | | 40% | 20% |

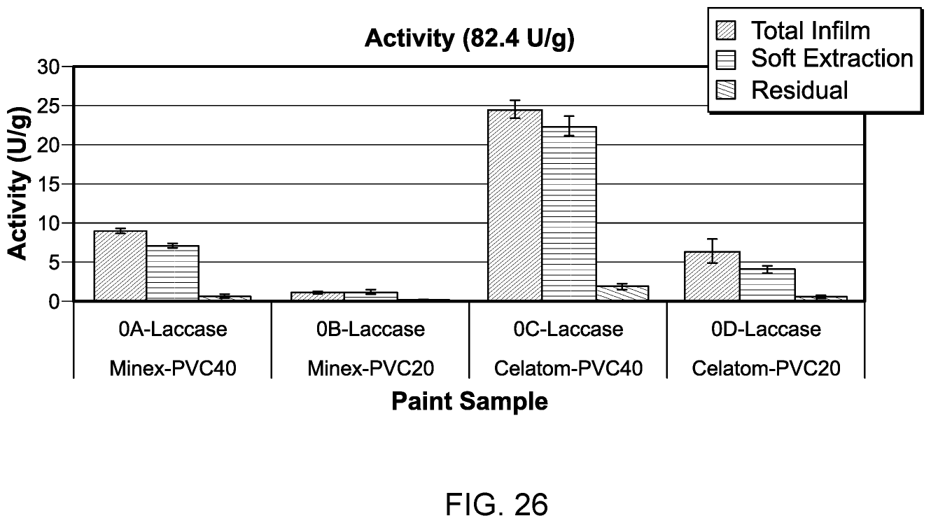

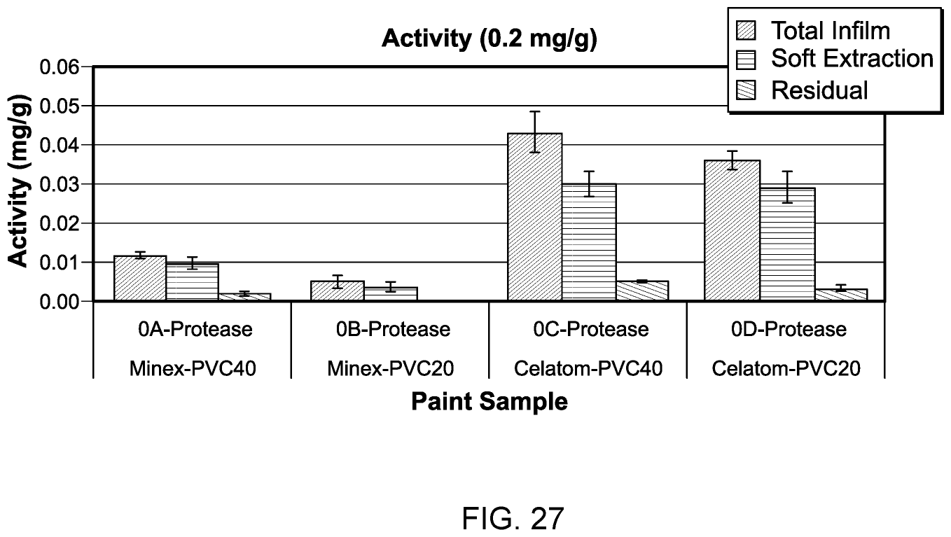

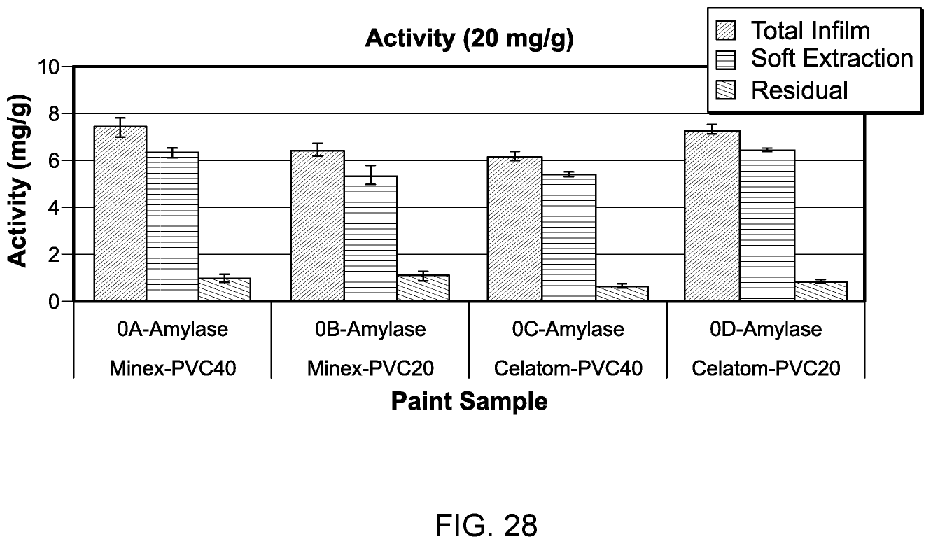

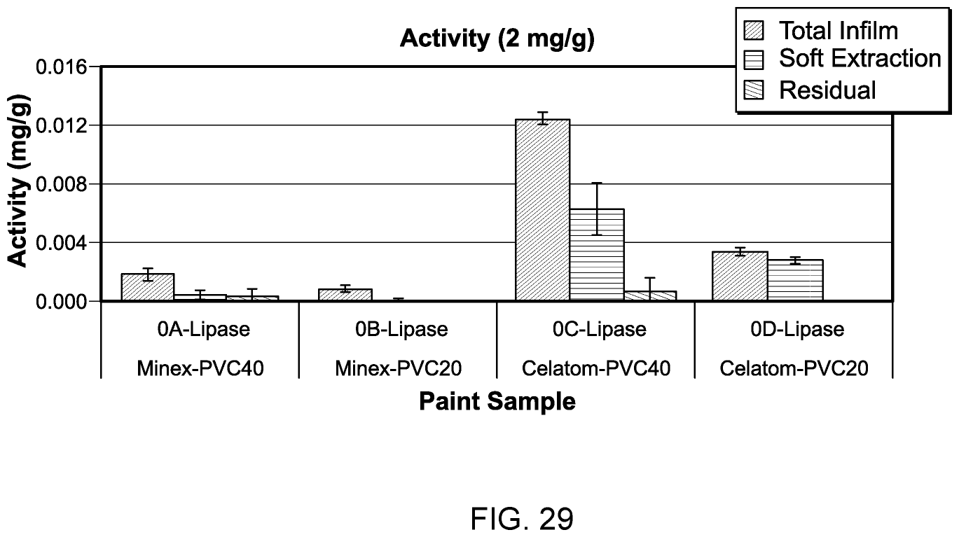

For paints embedded with laccase, protease, or lipase, substantially higher in-film enzyme activity was observed in Celatom-containing paints than that of Minex 4, and higher PVC levels resulted in higher in-film activity (FIGS. 26, 27 and 29). This is consistent with the in-film activity assays of Group 1 and Group 2 paint samples embedded with cellulase. The highest in-film activity (in Celatom-containing paint with PVC of 40%) measured at ˜30% for laccase, ˜20% for protease, and ˜6% for lipase, respectively, of added enzyme activity level. However, paints embedded with amylase exhibited similarly high in-film activity (˜30% of added enzyme activity level) across all paint formulations (FIG. 28).

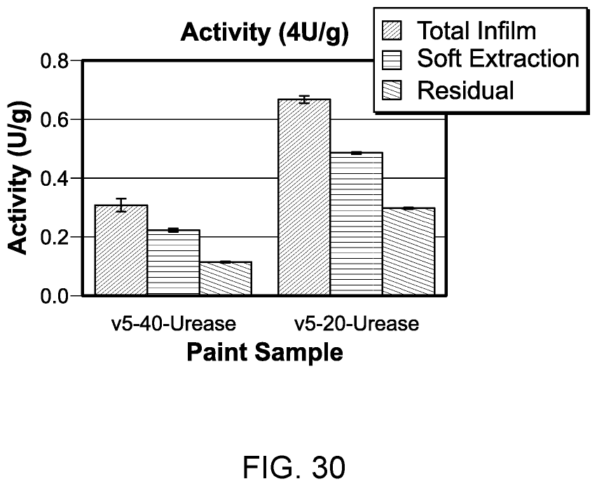

Unexpectedly, PVC levels had the opposite effect on urease in-film activity, with lower PVC levels resulting in higher in-film urease activity (FIG. 30). The highest in-film activity (in paint with PVC of 20%) measured at ˜20% of added urease activity level. For films embedded with laccase, protease, amylase, urease, or lipase, the majority of the in-film activity can be attributed to “soft” extracted enzyme, with very low residual activity detected.

### CONCLUSIONS

Both the agar plate assays and the in-film biochemical activity assays work unexpectedly well across a variety of enzymes classes and paint samples. Further, as validation of these methods, similar results were obtained by the other methods described herein across different paint formulations and enzyme classes. Configuring the film to allow light to pass through by, for example, removing an interior region, worked unexpectedly well and across a range of enzyme classes and paint formulations. These experiments provide proof-of-concept for the use of the agar assays and biochemical assays developed herein as screening tools. In-film enzyme activity, measured as % of added enzyme activity level, varied significantly among different enzyme classes. The majority of this in-film activity can be attributed to “soft” extracted enzyme, as residual film activity is very low. Finally higher PVC levels consistently result in higher in-film activity for most enzyme classes; however, urease showed an opposite trend; and paint with Celatom filler has higher in-film enzyme activity than paint with Minex 4 filler for most enzyme classes. Collectively, these studies provide further proof of principle that multiple classes of enzymes directly embedded in wet paint retain their activity, and further that they remain active following film formation.

### Example 6: In Situ Localization and Activity of Enzyme in Film

This example shows microscopic methods for the visualization of the in situ localization of enzyme in dry paint film and in wet paint, and the in situ activity of enzyme dry paint film. Another aim of these investigations was to discover the impact of paint formulation ingredients on the distribution of enzyme in the film and activity within film. Finally, these studies were conducted to provide further confirmation of the in-film activity of enzymes that was detected and measured by other assay methods.

### Visualization of In Situ Enzyme Activity in Film

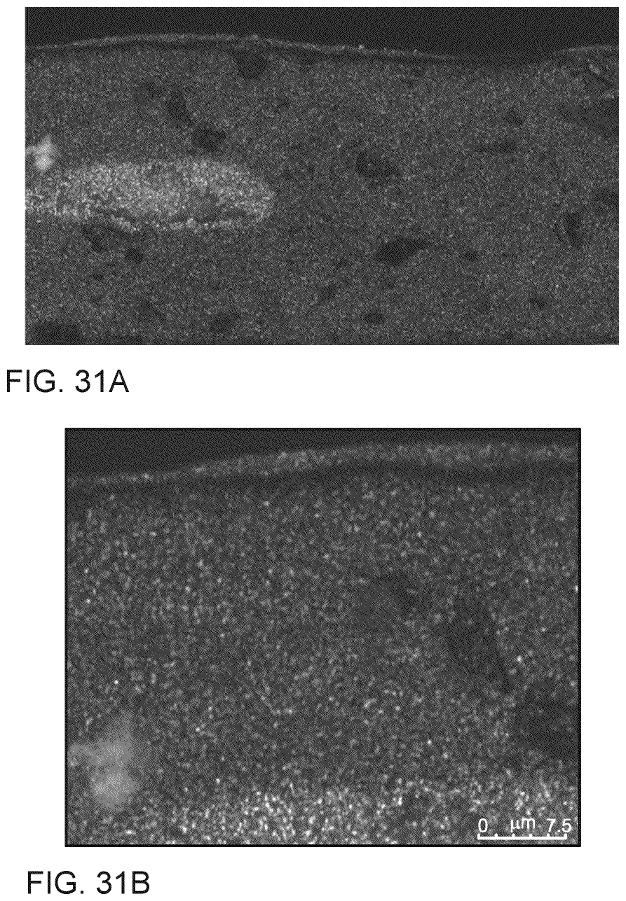

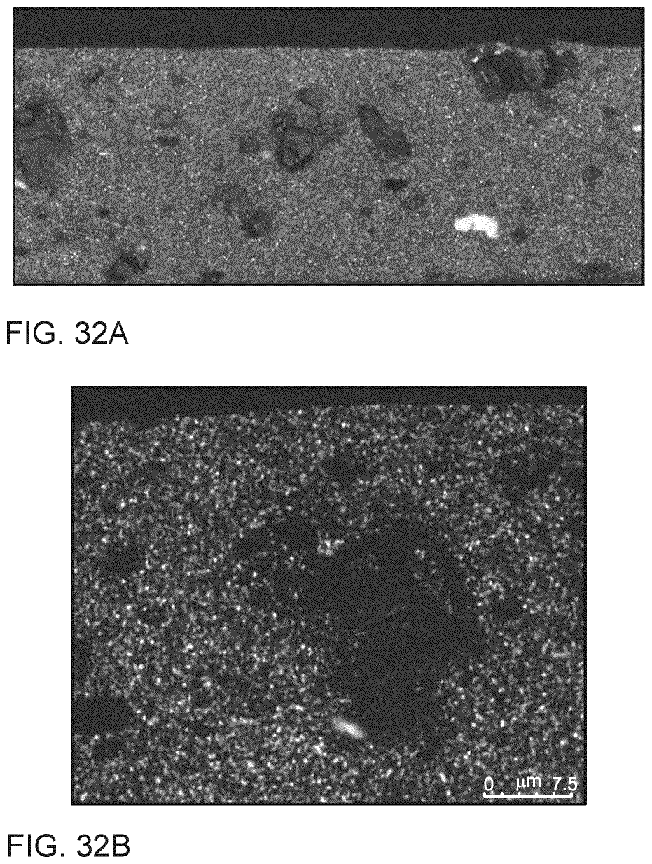

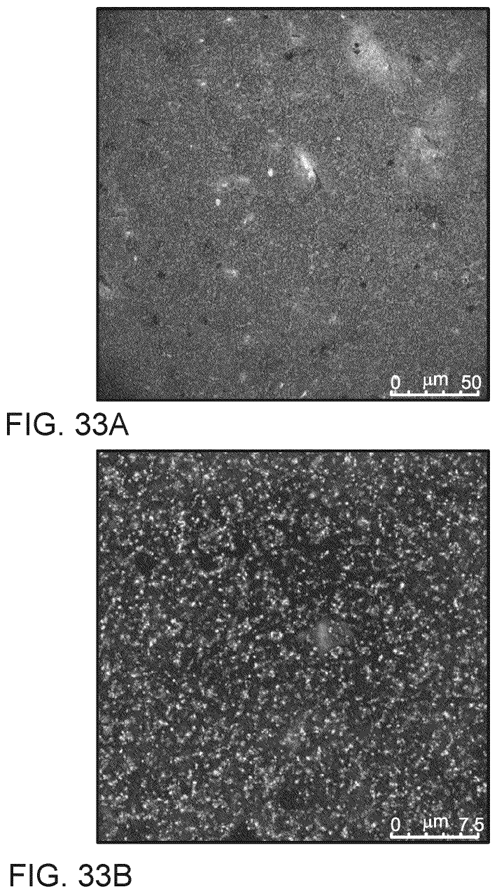

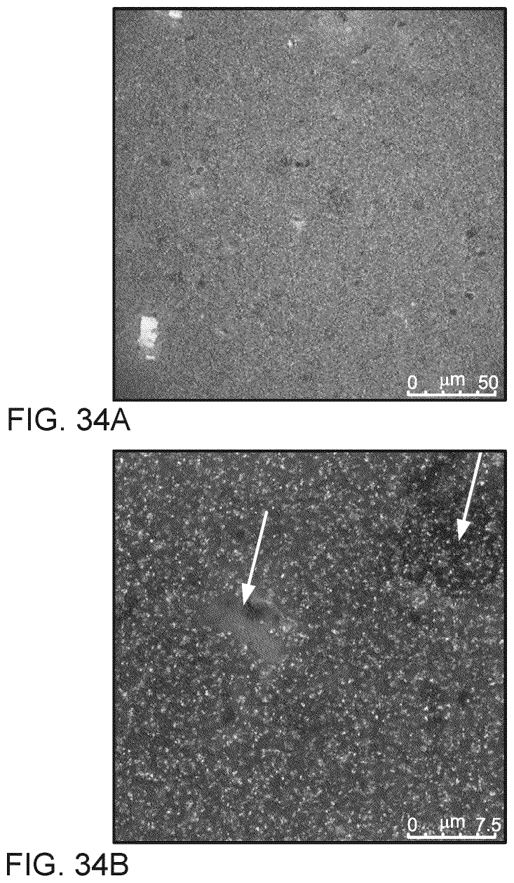

A cellulase enzyme (Pyrolase HT) was covalently labeled by a fluorescence dye (fluorescein), which was then added to liquid paint samples; paint films were drawn down and dried. The enzyme distribution was visualized in dry paint film (at both the bottom surface and at a cross section) using confocal laser scanning microscopy (CLSM). Both low and high magnification images were captured, where the grey color is due to light scattering from the TiO₂ pigment and the fluorescence glow is due to the fluorescently labeled enzyme. Microscopic analysis of a cross section of paint film comprising Minex filler [(NaK)Al₂(AlSi₃)O₁₀(OH)₂] revealed that enzyme distribution appeared as small particles and greater domains, possibly located on some filler particles (FIG. 31). FIG. 32 shows a cross section of paint film comprising Duramite filler (CaCO₃), where a slight gradient in the distribution of enzyme towards the surface was observed and the enzyme appeared to be located predominantly on filler particles. A homogenous fluorescence was observed over the whole image of a bottom view of paint film comprising Minex filler, with some bright areas seen (FIG. 33). CLSM visualization of bottom view of paint film comprising Duramite filler also yielded a homogenous fluorescence over the whole image (FIG. 34), with some areas which are free of enzymes and other areas where enzymes adsorb strongly (likely CaCO₃ filler) (indicated by arrows in FIG. 34B).

These microscopic analyses revealed a generally inhomogeneous distribution of enzyme in the dry paint samples. Enzymes appeared to migrate toward the surface of the film and form a gradient across the film. Adsorption onto the filler particles within the film and unspecified agglomerates was further observed. In liquid paint samples, the enzyme appears inhomogeneously distributed, and is predominantly in the water phase, which forms a separate phase besides a TiO₂/binder phase in liquid paint. No adsorption of enzyme on filler particles is observed in liquid paint, neither for the Minex nor for the Duramite fillers.

### Visualization of In Situ Enzyme Activity in Film

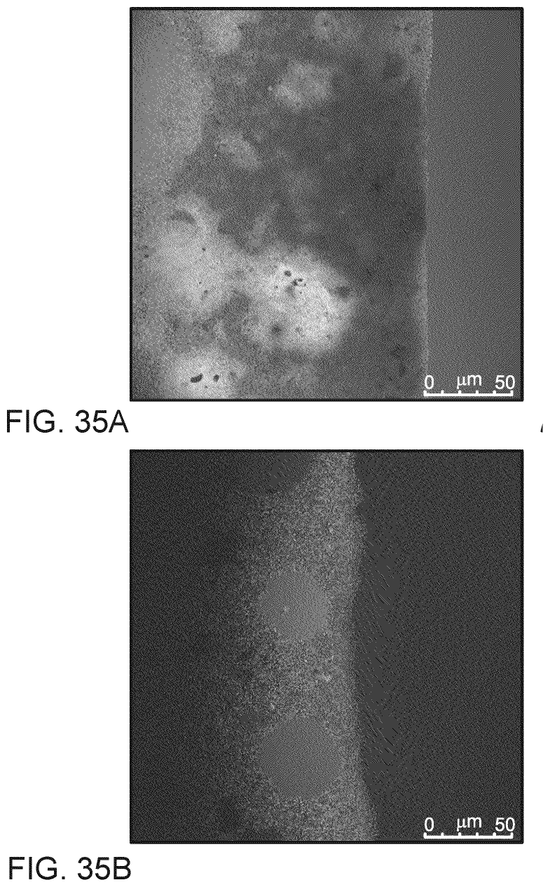

To visualize in-film enzyme activity, a substrate solution (Resorufin Cellubioside) was applied at the edge or cross section of paint film. A substrate solution (100 μmol Resorufin Cellubioside) was applied at the edge of the film, and as the substrate is converted by cellulase enzyme, released Resorufin dye fluoresces. FIG. 35 shows confocal laser scanning microscopy visualization of enzyme activity in a paint film comprising Minex filler [(NaK)Al₂(AlSi₃)O₁₀(OH)₂]. An overlay of reflection and fluorescence shows a grey reflection due to scattering from the TiO₂ pigment, while the fluorescence glow is due to the release of fluorescence dye Resorufin from substrate Resorufin Cellubioside by cellulase activity. Substrate conversion to product by the enzyme is observed within the film (FIG. 35). The converted product (dye) or the enzymes was seen to diffuse into the surrounding solution phase as well. Interestingly, penetration of fluorescence into the film is slower in the lower PVC sample (FIG. 35B).

In conclusion, the conversion of the substrate (Resorufin Cellubioside) by enzyme can be visualized in the paint film. Interestingly, the penetration of the substrate into the film and subsequent enzymatic conversion is faster in higher PVC sample. The released fluorescent dye from the enzymatic reaction, Resorufin, is enriched at the interface of the filler particle. However, the free dye molecule itself is slightly hydrophobic and also adsorbs stronger at interfaces of the filler particles. Therefore, one cannot conclude directly that the enzyme is located predominately at these interfaces. Collectively, these studies provide further proof of principle that multiple classes of enzymes directly embedded in wet paint retain their activity, and further that they remain active following film formation.

In at least some of the previously described embodiments, one or more elements used in one embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

1. A method of detecting of an enzyme activity in a coating composition, comprising the steps of:
(a) contacting the coating composition with a surface of a medium, wherein the coating composition comprises an enzyme and the medium comprises a substrate of the enzyme;
(b) incubating the medium that is contacted with the coating composition of (a) under a condition to allow the enzyme to react with the substrate in the coating composition; and
(c) monitoring one or more physical properties of the medium which is in contact with the coating composition;
whereby a change in at least one of the one or more physical properties of the medium indicates an activity of the enzyme.

2. The method of claim 1, wherein the coating composition comprises a film.

3. The method of claim 1, wherein the condition to allow the enzyme to react with the substrate comprises a period of time sufficient to allow the enzyme to react with the substrate, a suitable pH for enzyme activity, a suitable temperature, a suitable moisture level for enzyme activity, or a combination thereof.

4. The method of claim 1, wherein the one or more physical properties comprise a color property, an optical property, or a combination thereof, and optionally the optical property comprises opacity and/or transparency of the medium.

5. The method of claim 1, wherein the substrate is a chromogenic substrate, a fluorescent substrate and/or a luminescent substrate

6. The method of claim 1, wherein a product indicates the activity of the enzyme, and wherein the product is a chromogenic product, a fluorescent product, and/or a luminescent product.

7. The method of claim 1, wherein the medium further comprises an indicator dye;
wherein the indicator dye has one or more of the following properties: enhances contrast to facilitate monitoring the opacity of the medium, binds the substrate, binds a product of the enzymatic reaction, or is responsive to a change in the pH of the medium resulting from the activity of the enzyme; and
optionally the indicator dye is selected from the group comprising thionin, astrazon orange, astrazon blue, toluidine blue, methylene blue, acridine orange, pyronine-G, proflavine, azure A, phloxine B, cresyl violet, safranine O, neutral red, thioflavin T, fast red AL, methylene green, rhodamine B, rhodamine 6G, azure B, indoine blue, brilliant cresyl blue, 4′,6-diamidino-2-phenylindole dihydrochloride hydrate, acridine yellow, acriflavine, pyronin-Y, pyronin-B, meldola's blue, nile blue, nile red, new methylene blue, methyl violet, a triphenylmethane dye, methyl green, crystal violet, victoria blue, brilliant green, basic fuchsin, new fuchsin, ethyl violet, malachite green oxalate, quinaldine red, pinacryptol yellow, pinacyanol bromide, pinacyanol chloride, 2-[4-(dimethylamino)styrl]-1-methylquinolium iodide, 2-[4-(dimethylamino)styrl]-1-methylpyridinium iodide, stains-all, benzopurpurin, methyl green, chlorphenol Red, Bromocresol Green, Bromocresol Purple, Bromothymol Blue, Phenol Red, Thymol Blue, Cresol Red, Alizarin, Mordant Orange, Methyl Orange, Methyl Red, Reichardt's Dye, Congo Red, Eosin Blue, Fat Brown B, Orange G, Metanil Yellow, Naphthol Green B, Methylene Violet 3RAX, Sudan Orange G, Morin Hydrate, Disperse Orange 25, Rosolic Acid, Fat Brown RR, Cyanidin chloride, 3,6-Acridineamine, 6′-Butoxy-2,6-diamino-3,3′-azodipyridine, para-Rosaniline Base, Acridine Orange Base, Carbinol Base, and any combination thereof.

8. The method of claim 2, wherein the change in the one or more physical properties occurs in the medium underneath the film and/or in the medium surrounding the film.

9. The method of claim 2, wherein the film is not contacted with a liquid prior to step (a).

10. The method of claim 1, wherein the medium is substantially flat, and optionally the medium is selected from the group comprising agar, gelatine, poiyvinylalcohol, polyetherglycols, polyethylene glycol monostearate, diethylene glycol distearate, ester wax, polyester wax, nitrocellulose, paraffin wax, and any combination thereof.

11. The method of claim 1, wherein one or more of steps (a), (b), or (c) are assisted by automation.

12. The method of claim 1, wherein the coating composition comprises a paint, a lacquer, a printing ink, a varnish, a shellac, a stain, a textile finish, a sealing compound, a water repellent coating, or any combination thereof.

13. The method of claim 1, wherein the substrate is a natural substrate or a synthetic substrate, and wherein the substrate comprises milk, casein, azo-barley glucan, azo-carob galactomannan, p-nitrophenyl-B-D-lactopyranoside, red starch, syringaldazine, vegetable oil, azo-xylan, azo-arabinoxylan or any combination thereof.

14. The method of claim 1, wherein the enzyme is selected from the group comprising an amylase, a lipase, a protease, a laccase, a urease, a mannanase, a cellulase, a xylanase, a formaldehyde dismutase, a phytase, an aminopeptidase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, isomerase, a mannosidase, an oxidase, a pectinase, a peptidoglutaminase, a peroxidase, a polyphenoloxidase, a nuclease, a ribonuclease, a transglutaminase, a xylanase, a pullulanase, an isoamylase, a carrageenase, or any combination thereof.

15. A method of measuring an enzyme activity in a coating composition, comprising the steps of:
(a) configuring the coating composition to allow a spectrophotometer detection light to pass through the coating composition;
(b) placing the coating composition in a sample well of a spectrophotometer, wherein the coating composition comprises an enzyme and the sample well comprises a reaction buffer and a substrate of the enzyme; and
(c) monitoring the absorbance at a wavelength under a condition to allow the enzyme to react with the substrate;
whereby a change in the absorbance at the wavelength indicates an activity of the enzyme.

16. The method of claim 15, wherein the coating composition comprises a film, and optionally the film weighs about 1 mg to about 200 mg, and wherein the condition to allow the enzyme to react with the substrate comprises a period of time sufficient to allow the enzyme to react with the substrate, a suitable pH for enzyme activity, a suitable temperature for enzyme activity, or a combination thereof.

17. (canceled)

18. The method of claim 16, wherein step (a) comprises removing an interior region from the film, wherein the interior region is substantially circular, or other geometrical shape that allows that light pass through the central region of the film.

19. (canceled)

20. The method of claim 15, wherein the sample well is contained within a multi-well plate comprising a plurality of sample wells, and optionally the multi-well plate is selected from the group comprising a 6-well microplate, a 12-well microplate, a 24-well microplate, 96-well microplate, and 384-well microplate.

21. The method of claim 15, wherein step (c) is performed at a temperature of about 4° C. to about 80° C., and wherein step (c) is performed at one or more intervals for a time period of about 2 minutes to about 48 hours.

22. (canceled)

23. The method of claim 15, wherein the coating composition comprises a paint, a lacquer, a printing ink, a varnish, a shellac, a stain, a textile finish, a sealing compound, a water repellent coating, or any combination thereof thereof,
wherein the substrate is a natural substrate or a synthetic substrate, wherein the substrate is selected from the group comprising formaldehyde, syringaldazine, 2,2′-Azino-bis(3-Ethylbenzthiazoline-6-Sulfonic Acid), urea, 2-chloro-4-nitrophenyl-maltotrioside, Ala-Ala-Pro-Phe-p-nitrophenyl, p-nitrophenyl-Octanoate, 4-Nitrophenyl-β-D-cellobioside, formaldehyde, azo-carob galactomannan, p-nitrophenyl-B-D-lactopyranoside, azo-carob galactomannan, or p-nitrophenyl-B-D-lactopyranoside or any combination thereof,
and wherein the enzyme is selected from the group comprising an amylase, a lipase, a protease, a laccase, a urease, a mannanase, a cellulase, a xylanase, a formaldehyde dismutase, a phytase, an aminopeptidase, a carbohydrase, a carboxypeptidase, a catalase, a chitinase, a cutinase, a cyclodextrin glucanotransferase, a deoxyribonuclease, an esterase, an α-galactosidase, a β-galactosidase, a glucoamylase, α-glucosidase, a β-glucosidase, a haloperoxidase, an invertase, isomerase, a mannosidase, an oxidase, a pectinase, a peptidoglutaminase, a peroxidase, a polyphenoloxidase, a nuclease, a ribonuclease, a transglutaminase, a xylanase, a pullulanase, an isoamylase, a carrageenase, or any combination thereof.

24. (canceled)

25. (canceled)
